(19) United States
(12) Patent Application Publication
(10) Pub. No.: US 2021/0375409 A1
(43) Pub. Date: Dec. 2, 2021

(54) SYSTEMS AND METHODS FOR BLOCKCHAIN-BASED HEALTH DATA VALIDATION AND ACCESS MANAGEMENT

(71) Applicant: LONGENESIS LTD., Wan Chai (CN)

(72) Inventors: Konstantin ROMANTSOV, Pyatiletok (RU); Garri ZMUDZE, Lugano (CH); Aleksandrs ZAVORONKOVS, London (GB)

(21) Appl. No.: 17/286,721

(22) PCT Filed: Mar. 22, 2019
(86) PCT No.: PCT/IB2019/052363

§ 371 Date: Apr. 19, 2021

Related U.S. Application Data
(60) Provisional application No. 62/747,943, filed on Oct. 19, 2018.
(60) Provisional application No. 62/758,041, filed on Nov. 9, 2018.

(51) Int. Cl. G16H 10/60; G06F 21/62; G06F 21/60

(52) U.S. Cl. CPC G16H 10/60; G06F 21/602; G06F 21/6245

(57) ABSTRACT

A system for blockchain-based storage and access control of health records is provided. The system may include a health data storage configured to store health data items, which may be associated with a plurality of users. The system may also include a user access portal configured to transmit an uploaded health data item to the health data storage. The user access portal may also add a data upload transaction to a blockchain identifying a storage location of the uploaded health data item within the health data storage.

### BACKGROUND

The digital revolution in medicine produced a paradigm shift in the healthcare industry. One of the major benefits of the digital healthcare system and electronic medical records is improved access to the healthcare records for both health professionals and patients. The success of initiatives that provide patients with the access to their electronic healthcare records, such as OpenNotes, suggests their potential to improve the quality and efficiency of medical care.

However, biomedical data is not limited to the clinical records created by physicians. For example, a substantial amount of data is retrieved from biomedical imaging, laboratory testing such as basic blood tests, and omics data. Notably, the amount of genomic data alone is projected to surpass the amount of data generated by other data-intensive fields such as social networks and online video-sharing platforms.

National healthcare programs such as the UK Biobank (supported by the National Health Service (NHS)) and global programs such as the Library of Integrated Network-Based Cellular Signatures (LINCS) Consortium and the Encyclopedia of DNA Elements (ENCODE) Project provide scientists with tens of thousands of high-quality data samples. However, while increased data volume and complexity offers new exciting perspectives in healthcare industry development, it also introduces new challenges in data analysis and interpretation, and of course, privacy and security. Due to huge demand for the treatments and prevention of chronic diseases, mainly driven by an aging population, there is a need for new global integrative healthcare approaches.

### SUMMARY

The present disclosure presents new and innovative systems and methods incorporating blockchain technology into the upload, storage, and tracking of user health data. In one embodiment, a system is configured to create and use a blockchain to store and control access to data uploaded by users (e.g., patients). The system may also validate the uploaded data for accuracy and completeness after upload and provide data scoring tools for uploaded data analysis and report generation (e.g. risk factor analysis). Interested parties (e.g., research institutions, medical companies) may then purchase or request access to the health data via the blockchain for use in research and product development.

In another embodiment, a system is provided comprising a health data storage configured to store health data items associated with a plurality of users and a user access portal configured to transmit an uploaded health data item to the health data storage and add a data upload transaction to a blockchain identifying a storage location of the uploaded health data item within the health data storage.

In yet another embodiment, the blockchain is implemented by a plurality of nodes that verify transactions using a consensus algorithm before storing the transactions on the blockchain.

In a further embodiment, the system further comprises a validator configured to perform an upload validation operation on the uploaded health data item and to add an upload validation transaction to the blockchain reflecting a result of the upload validation operation performed on the uploaded health data item.

In a still further embodiment, the validator is configured to analyze the uploaded health data item with the upload validation operation to verify a data quality of the uploaded health data item before storing the uploaded health data item in the health data storage.

In another embodiment, the system further comprises a customer portal configured to grant and remove a customer access to a purchased health data item from the stored health data items.

In yet another embodiment, the customer portal is further configured to generate a permission assignment transaction identifying (i) the customer, (ii) the purchased health data item, and (iii) an access permission level granted to the customer for the purchased health data item.

In a further embodiment, the system further comprises a key management system configured to (i) split a private key associated with the uploaded health data item into a plurality of key parts and (ii) store the plurality of key parts on a plurality of key holders, wherein the private key is used to encrypt the uploaded health data item to create an encrypted health data item before storing the encrypted health data item within the health data storage.

In a still further embodiment, the key management system is further configured to (i) combine the key parts to reconstruct the private key and (ii) decrypt the encrypted health data item upon receiving a download request for the uploaded health data item.

In another embodiment, the health data storage is also configured to store access permission levels granted to one or more users for one or more of the health data items.

In yet another embodiment, the access permission levels include one or more permissions from the group consisting of: (i) read access to the stored health data items, and (ii) read access to data resulting from calculations performed on the stored health data items.

In a further embodiment, a method is provided comprising receiving a health data item, storing the health data item on a health data storage, generating a data upload transaction indicating the health data item and a user associated with the health data item, and storing the data upload transaction on a blockchain.

In a still further embodiment, the method further comprises verifying the data upload transaction with a plurality of nodes, wherein the nodes are configured to implement the blockchain.

In another embodiment, the method further comprises validating a data quality of the health data item with an upload validation operation, generating an upload validation transaction indicating the health data item and an upload validation result of the upload validation operation, and storing the upload validation transaction on the blockchain.

In yet another embodiment, the method further comprises receiving a data access request from a customer requesting access to the health data item, generating a permission assignment transaction indicating the customer, the health data item, and a permission level granted to the customer, and storing the permission assignment transaction on the blockchain.

In a further embodiment, the method further comprises comparing the data access request to a privacy setting of the user, and determining that the data access request complies with the privacy setting of the user.

In a still further embodiment, the data access request includes a request for a plurality of health data items meeting one or more request criteria.

In another embodiment, the method further comprises retrieving the health data item from the health data storage, validating the health data item with a download validation operation, generating a download validation transaction indicating the health data item, the customer, and a download validation result, and storing the download validation transaction on the blockchain.

In yet another embodiment, the health data item is encrypted on the health data storage. In such embodiment, the method may also further comprise decrypting the health data item, encrypting the health data item with an encryption key associated with the customer to create an encrypted health data item, and providing the encrypted health data item to the customer.

In a further embodiment, the method further comprises receiving a private key associated with the uploaded health data item, splitting the private key into a plurality of key parts, and storing the plurality of key parts in a plurality of key holders.

In a still further embodiment, the method further comprises retrieving the plurality of key parts from the key holders and reconstructing the private key from the plurality of key parts.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 illustrates a block diagram of a system according to an exemplary embodiment of the present disclosure.

FIG. 2 illustrates a block diagram of a key management system according to an exemplary embodiment of the present disclosure.

FIG. 3 illustrates a block diagram of a blockchain-based health data storage system according to an exemplary embodiment of the present disclosure.

FIG. 4 illustrates a plurality of transactions according to exemplary embodiments of the present disclosure.

FIGS. 5A-5B illustrate flowcharts of methods for data upload according to exemplary embodiments of the present disclosure.

FIG. 6 illustrates a flowchart of a method for providing data access according to an exemplary embodiment of the present disclosure.

FIG. 7 illustrates a flowchart of a method for validating health data items according to an exemplary embodiment of the present disclosure.

FIGS. 8A-8B illustrate flowcharts of methods for managing encryption keys according to exemplary embodiments of the present disclosure.

FIGS. 9A-9C illustrate a plurality of methods 900, 940, 972 according to exemplary embodiments of the present disclosure.

FIG. 10 illustrates a blockchain of a system according to an exemplary embodiment of the present disclosure.

FIGS. 11 and 12A-12B illustrate a plurality of data valuation graphs according to an exemplary embodiment of the present disclosure.

### DETAILED DESCRIPTION OF EXAMPLE EMBODIMENTS

### Overview

Many recent approaches to personalized medicine in oncology and other diseases rely on the various types of health data, including genomic, transcriptomic, microRNA, proteomic, antigen, methylation, imaging, metagenomic, mitochondrial, metabolic, physiological, and other data. And while several attempts have been made to evaluate the clinical benefit of the different methods, including the use of multiple data types for evaluating the health status of individuals, prior approaches are not truly integrative on the population scale and fail to compare the predictive nature and value of the various data types in the context of biomedicine. Introduction of new technologies, such as artificial intelligence and blockchain, may enhance and scale up the progress in health care sciences and lead to effective and cost-efficient healthcare ecosystems.

While the amount of health-associated data and the number of large scales global projects increases, integrative analysis of this data is still presenting issues. Even high-quality biomedical data is usually highly heterogeneous and complex and requires special approaches for preprocessing and analysis. Computational biology methods are routinely used in various fields of healthcare and are incorporated in pipelines of pharmaceutical companies. Machine learning techniques, such as deep neural networks (DNNs) are among the leading and the most promising tools of computational analysis and are able to capture high-level dependencies in the healthcare data. For example, DNNs are showing promise to predict, e.g., drug properties, biomarkers, and patient health. Similarly, convolutional neural networks (CNNs) show promise in classifying cancer patients using immunohistochemistry of tumor tissues.

There are many promising machine learning techniques in practice and in development including the upcoming capsule networks and recursive cortical networks and many advances are being made in symbolic learning and natural language processing. However, the recurrent neural networks, generative adversarial networks (GANs), and transfer learning techniques (such as one and zero-shot learning techniques) are gaining popularity in the healthcare applications and are particularly compatible with distributed health data storage solutions, such as blockchain-enabled personal health data marketplaces. Notably, many of these machine learning techniques require extensive amounts of data, which may be difficult for a centralized system or entity to gather on its own, which may impede the progress of such medical research.

However, collecting the required large amounts of medical data is not without its issues. A high percentage of personal health data generated by individuals could be considered private. To ensure propriety in the handling of data, there have been regulations and rules that guide processes including generation, use, transfer, access, and exchange of data. Such regulations include the Health Insurance Portability and Accountability Act (HIPAA) of 1996 and Privacy Rule's minimum necessary standard and the European Union's General Data Protection Regulation (GDPR), which includes requirements that users be able to delete and control the usage of private data. Additionally, most people believe that their medical and other health information is private and should be protected, and patients usually want to know how this information is being handled.

One such method to address the above-identified need for easier and larger-scale personal health data collection and storage is to provide a blockchain-enabled system to receive, validate, and control access to personal health data items uploaded by one or more users in a safe and secure fashion. For example, such a blockchain-enabled system may include a health data storage and is configured to store health data items uploaded by a plurality of users. The health data items stored on the health data storage may be tracked and monitored on a public or private ledger, such as a blockchain implemented by a plurality of nodes. The blockchain may enable public audits of access to the health data items uploaded by the users. For example, the blockchain may allow for assignment and revocation of access permissions to one or more customers, such as individuals or institutions performing medical research, and for increased auditability and traceability of operations on the health data items. Additionally, such a blockchain-enabled health data storage system may enable a personal health data marketplace by allowing one or more customers to exchange an incentive mechanism with users that have uploaded health data items in exchange for receiving access to those health data items. Such a publicly-accessible system may also allow individuals to search the uploaded health data items in order to determine whether there are sufficient health data items of a certain type required for the individuals' research.

Also, by implementing personal health data storage on a blockchain, access and reviewability by external auditors (e.g., regulators, non-government organizations, law enforcement) is improved. This system would improve auditors' ability to verify the correctness of transaction processing in real time and/or retrospectively. Auditors may also be able to store a replica of the entire blockchain or a portion of the blockchain and may thus be able to quickly perform complete audits.

However, because the system may allow for members of the general public to upload health data items, it may be necessary to validate the accuracy and relevancy of uploaded health data items. Accordingly, the system may leverage the subject matter expertise of specialized organizations through the use of validators that may validate uploaded health data items. Additionally, to ensure accuracy of health data items downloaded from the system, it may further be necessary to validate the health data items prior to their download by the customer or individual who has received access to the health data items. For example, it may be necessary to validate health data items prior to download to ensure that the health data items are not corrupted.

Further, in order to maintain required security and to comply with the above-discussed regulatory requirements, the health data items may be transmitted and stored in an encrypted fashion. Therefore, it may be necessary to implement a key management system configured to securely process and store the encryption keys used to encrypt the health data items prior to their uploaded to the system and to decrypt the health data items for access by, e.g., research institutions and other customers. Such a system may split the encryption keys used to encrypt the health data items into multiple key parts and may separately store and encrypt each key part on a separate key holder connected to the system.

### System Description

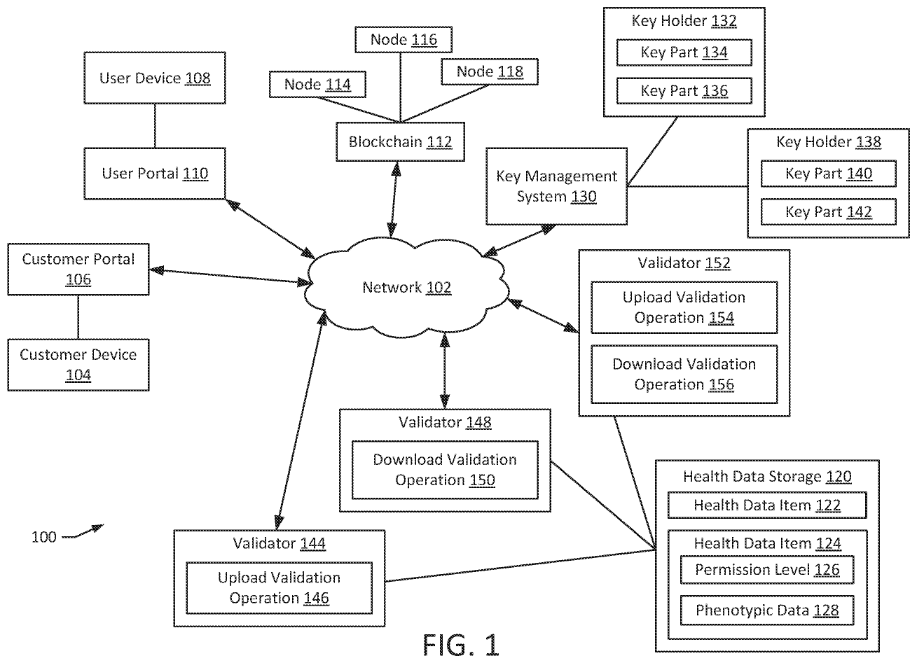

FIG. 1 depicts a block diagram of a system 100 according to an exemplary embodiment of the present disclosure. The system 100 may be used to upload and store health data from a plurality of users. The system 100 may also provide data validation services to validate the completeness and/or accuracy of uploaded health data. Further, the system 100 may service one or more customers who purchase or request access to the uploaded health data for use in research and other contexts.

The system 100 includes a customer portal 106 associated with the customer device 104, a user portal 110 associated with a user device 108. The system also includes a blockchain 112 implemented by a plurality of nodes 114, 116, 118. The blockchain is associated with a health data storage 120 storing multiple health data items 122, 124. As depicted, the health data item 124 includes a permission level 126 and a phenotypic data 128. The system 100 further includes a key management system 130 associated with key holders 132, 138 storing key parts 134, 136, 140, 142. The system 100 also includes a plurality of validators 152, 148, 144. As depicted, the validators 152, 148, 144 may include an upload validation operation 146 (e.g., validator 144), a download validation operation 150, (e.g., validator 148) or both an upload validation operation hundred and 54 and a download validation operation 156 (e.g., validator 152).

The customer portal 106, user portal 110, blockchain 112 key management system 130, and validators 144, 148, 152 are connected to a network 102. The network 102 may be implemented as a public network such as the Internet or may be implemented as a private network such as a private internal network for, e.g., a business, government agency, hospital. As another example, the system 100 may be implemented across a plurality of public and private health clinics. The system 100 may be used to help an organization set and deploy an ecosystem that includes a traceable and accountable data management system across all of their clinics. These systems may also be used within clinics that collaborate with pharmaceutical organizations, e.g., for clinical trial management, patient sourcing based on phenotypic data, patient follow-ups, collecting and analyzing patient health data, doctor identification, patient consent management, and tracing processes across the organization.

The user portal 110 may provide access to the system 100 (e.g., the blockchain 112 and the health data storage 120). The user portal 110 may be associated with the user device 108, such as a smart phone, laptop, tablet computer, or other computing device. For example, the user portal 110 may be an application installed on the user device 108 or may be implemented as a webpage accessible via the user device 108. A user may interact with the user portal 110 to upload one or more health data items 122, 124 to a health data storage 120, as discussed further below. For example, the user portal 110 may generate a data upload transaction for storage on the blockchain 112. Similarly, a user may use the user portal 110 to modify or adjust one or more privacy settings associated with health data items 122, 124 previously uploaded to the health data storage 120, or to delete previously-uploaded health data items 122, 124. For example, the user portal 110 may generate a permission assignment transaction for storage on the blockchain 112. A user may also use the user portal 110 to view a health data dashboard, which may include, e.g., an overview of uploaded health data items 122, 124, an analysis of uploaded health data items 122, 124 (e.g., to identify one or more risk factors), and information regarding any incentive received for uploading health data items 122, 124.

The customer portal 106 may provide access to the system 100 (e.g., the blockchain 112, the health data storage 120). Like the user portal 110, the customer portal 106 may be associated with a customer device 104, such as a smart phone, laptop, tablet computer, or other computing device and may be an application or other piece of software installed on the customer device 104. For example, the customer portal 106 may be an application installed on the customer device 104, or may be implemented as a webpage accessible via the customer device 104. The customer portal 106 may be used to search for and request access to one or more health data items 122, 124 stored on the health data storage 120. For example, the customer portal 106 may enable user to search for one or more health data items 122, 124 that meet certain specified conditions (e.g., conditions regarding the user uploaded the health data items 122, 124, conditions regarding the content or type of the health data item 122, 124, health data items 122, 124 associated with individuals having one or more medical conditions). For example, the customer portal 106 may be configured to generate a smart contract for execution on the blockchain 112 and may generate one or more permission assignment transaction adjusting access permission levels for the associated customer to enable access to the desired health data items 122, 124 on the health data storage 120. In certain implementations, the customer portal 106 and the user portal 110 may be implemented by the same piece of software (e.g., the same application on a user device 108 or a customer device 104).

The blockchain 112 may be configured to store one or more transactions, such as data upload transactions reflecting health data items 122, 124 uploaded to the health data storage 120, permission assignment transaction reflecting the assignment of permission to access by a customer for one or more health data items 122, 124, upload validation transactions reflecting the result of one or more upload validation operations performed on health data items 122, 124 uploaded to the health the storage 120, and download validation transactions reflecting the result of a download validation operation performed on health data items 122, 124 downloaded from the health data storage (e.g., by a customer who has been granted access).

The blockchain 112 may be implemented by a plurality of nodes 114, 116, 118. The nodes 114, 116, 118 may be configured to verify and store incoming transactions (e.g., data upload transactions, permission assignment transactions, upload validation transactions, download validation transactions) using a consensus algorithm. For example, the blockchain 112 may verify and store incoming transactions using a Byzantine Fault Tolerance (BFT) consensus algorithm, or a proof-of-work consensus algorithm. In certain implementations, the blockchain 112 may be implemented as a private blockchain, and the nodes 114, 116, 118 may be networked on a private network, and in certain implementations may be networked via a network separate from the network 102. For example, the nodes 114, 116, 118 may communicate via a private network, while the network 102 may be implemented by the Internet.

The health data storage 120 may be configured to store health data items 122, 124 uploaded by users via the user portal 110. As depicted, the health data item 124 includes phenotypic data 128 which may include a collection of characteristics of the user (e.g., height, weight, demographic data, disease symptoms, relevant health conditions). The health data item 124 also includes a permission level 126, which may indicate a level of access granted to one or more customers using the system 100. For example, the permission level 126 may indicate that a particular customer is provided read-only access to the health data item 124, is allowed to copy raw data from the health data item 124 or is only able to access data resulting from calculations performed on the health data item 124. The granted level of access may differ between individual customers, so the permission level 126 may include a separate entry for each customer that whose access to the health data item 124 has been controlled. Although not depicted, health data item 122 may include information similar to the permission level 126 and the phenotypic data 128.

The health data storage 120 is associated with the blockchain 112, as indicated in FIG. 1. This association may indicate that transactions stored on the blockchain 112 include information relating to health data items 122, 124 stored on the health data storage 120. For example, transactions (e.g., data upload transactions) may include an identifier for the health data item 122, 124 for which the transaction is applicable. In another example, a permission assignment transaction may identify the health data item 122, 124 for which access is being granted. Users and customers accessing the blockchain 112 via the customer portal 106 and/or the user portal 110 may then be able to determine the access level granted to the customer for the relevant health data item 122, 124. The health data storage 120 is depicted as connected to the validator's 144, 140, 152. The connection to the validator's 144, 140, 152 may preferably be implemented on a private network, such as the same or similar private network to the private network connecting the nodes 114, 116, 118 in certain implementations. Such a configuration may be selected to, e.g., increase security by prohibiting direct access to the health data storage 125 via the network 102. However, in other implementations, the health data storage 120 may be connected directly to the network 100 and to, such as where security concerns are less of a priority, or where deployment requirements (e.g., user convenience, access speed, performance requirements) necessitate a direct connection to the network 100 and to in the health data storage 120.

The key management system 130 may be configured to manage the encryption keys used to encrypt the health data items 122, 124 stored on the health data storage 120. For example, prior to uploading a health data item 122, 124 to the health data storage 120, the user portal 110 may encrypt the health data item 122, 124 using an encryption key associated with the user. In order for a customer to access this data, the health data item 122, 124 may have to be decrypted using the user's encryption key. Therefore, to enable later access to the stored health data items 122, 124, the key management system 130 may store the encryption keys. As will be discussed further below, the key management system 130 may split the encryption keys into multiple key parts 134, 136, 140, 142 and may store those key parts 134, 136, 140, 142 on key holders 132, 138. In certain implementations, the key management system may store a single key part 134, 136, 140, 142 corresponding to the user's encryption keys on each key holder 132, 138. For example, a user encryption key may be split into two key parts 134, 140, which are separately stored on the two key holders 132, 138.

The validators 144, 140, 152 may be configured to validate health data items 122, 124 uploaded to the health data storage 120, and downloaded or accessed by customers. For example, the validators may be configured to check a data quality of uploaded health data items 122, 124, or may be configured to confirm that health data items 122, 124 that are accessed or downloaded are not corrupted. In certain implementations, the validator 144, 152 may include an upload validation operation 146, 154 that is performed on an uploaded health data item 122, 124 to check a data quality of an uploaded health data item 122, 124. For example, the upload validation operation 146, 150 for may be performed on the uploaded health data item 122, 124 to verify a data format or completeness measurement of the uploaded health data item 122, 124. In another example, the completeness measurement may include a measurement of a percentage of pieces of information (e.g., height, weight, blood pressure) expected to be in a health data item 122, 124 of a particular type (e.g., an annual physical). In certain implementations, the validator 148, 152 may include a download validation operation 150, 156. The download validation operation may be performed on a downloaded or accessed health data item 122, 124 to validate that the health data item 122, 124 has not been corrupted. For example, the download validation operation 150, 156 may determine whether the accessed health data item is stored within the health data storage 120. In another example, the download validation operation 150, 156 may download and decrypt the accessed health data item 122, 124 from the health data storage 120 and may provide read-only access to the customer. In a further example, the download validation operation 150, 156 may download and decrypt the accessed health data item 122, 124 and may re-encrypt the accessed health data item with an encryption key associated with the customer. In yet another example, the download validation operation 150, 156 may include downloading and decrypting the accessed health data item 122, 124 and checking the accessed health data item for corruption (e.g., using a checksum stored in a phenotypic data item 128). In implementations where the blockchain 112, nodes 114, 116, 118, health data storage 120, and key management system 130 are provided by a single entity (e.g., a business or foundation), the validators 144, 148, 152 may not be implemented by that entity. For example, if the above identified elements are implanted by a single entity, the validators 144, 140, 152 may be implement by other entities with greater experience validating health data items 122, 124 (e.g., research institutions, hospitals, medical companies). As depicted, certain validators 148 may only implement a download validation operation 150, of the validators 144 may only implement upload validation operation 146, and still further validators 122 may implement both a download validation operation 156 and an upload validation operation 154.

In certain implementations, the validators 144, 148, 152 may additionally or alternatively be configured to score health data items 122, 124. In particular, the validators 144, 148, 152 may include one or more data scoring operations that include, e.g., statistical analysis models and artificial intelligence (AI) developed models for analyzing health data items 122, 124. The data scoring operations may analyze the health data items 122, 124 for one or more risk factors of the uploading user. For example, the data scoring operations may analyze genomic data in correlation with blood biomarkers and activity data to identify one or more heart disease risk factors. As another example, the data scoring operations may include AI-developed models that analyze photographs or scans of skin in connection with a equivalent biological age calculation. The results of the data scoring operations (e.g., a report presenting the results) may be viewable by a user via the user portal.

One or more of the system 100 features may be implemented with a CPU and/or a memory. For example, the customer portal 106, user portal 110, blockchain 112, key management system 130, health data storage 120, key holders 132, 138, and validators 144, 148,152 may be implemented with a CPU and/or a memory. In another example, the memory implementing one of the above features may contain instructions which, when executed by the CPU, may perform one or more of the features.

In yet another example, one or more of the components of the system 100 may be implemented by separate computing devices. For example, the customer device 104 associated with the customer portal 106, the user device 108 associated with the user portal 110, the nodes 114, 116, 118 implementing the blockchain 112, the health data storage 120, the key management system 130, the key holders 132, 138, and the validators 144, 148, 152 may be implemented as more than one computing device. For example, each of the above elements may be implemented as a separate computing device. In such implementations, the computing device implementing each of the above elements may include a CPU and memory that are configured to implement the features of the elements, as discussed above.

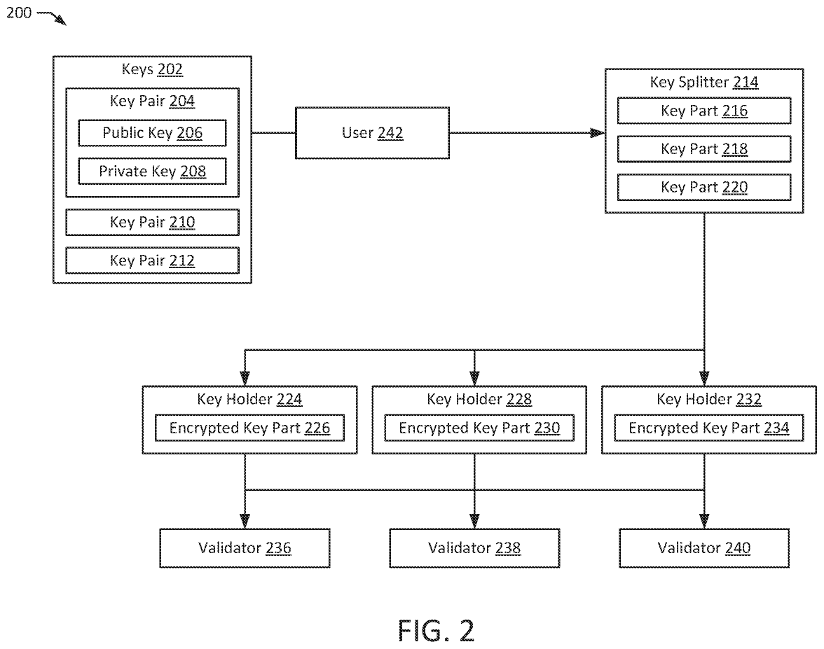

FIG. 2 depicts a block diagram of a key management system 200 according to an exemplary embodiment of the present disclosure. In certain implementations, the key management system 200 may implement, e.g., the key management system 130 and the key holders 132, 138 depicted of the system 100. The system 200 includes a user 242 associated with keys 202, a key splitter 214, key holders 224, 220, 232, and validators 236, 238, 240 (e.g., via a user device 108).

The user device 108, as described above, may include a smart phone, tablet computer, laptop, or other computing device and may include a user portal 110. The keys 202 associated with the user 242 may include key pairs 204, 210, 212 used to encrypt one or more health data items 122, 124 uploaded to a health data storage 120 via the user portal 110. In certain implementations, the user portal 110 may generate and/or store the keys 202 locally on the user device 108 and/or in the user portal 110. To provide access to the health data items 122, 124 uploaded to the health data system 120, it may be necessary to provide or otherwise utilize the key pairs 204, 210, 212 used to encrypt the health data items before upload to a party attempting to access the health data items 122, 124. For example, in certain implementations, the health data items 122, 124 may be encrypted using a symmetric encryption scheme, where the same key pair 204, 210, 212 is used to encrypt and decrypt the health data item 122, 124. In other implementations, the health data items 122, 124 may be encrypted using an asymmetric encryption scheme, where a different key pair 204, 210, 212 is used to decrypt the health data items 122, 124 than the key pair 204, 210, 212 used to encrypt the health data items 122, 124. In both symmetric and asymmetric encryption implementations, the key management system 200 may be used to store the key pairs 204, 210, 212 required to decrypt the health data items 122, 124.

Therefore, the key management system 200 may be used to securely manage and manipulate the key pairs 204, 210, 212. For example, the health data items 122, 124 may be encrypted using both a public key 206 and a private key 208 the public key 206 may be publicly available (e.g., available within a data upload transaction stored on the blockchain 112). However, the private key 208 may not be publicly available, but may be necessary to decrypt the health data item 122, 124. Therefore, the key management system 200 may be used to securely store the private key 208 and use the private key 208 to decrypt the encrypted health data item 122, 124 upon retrieval from the health data storage 120. In certain implementations, the health data storage 120 may allow for encrypting the health data items 122, 124 on a per user basis and/or on a per health data item basis. In implementations where the health data storage 120 stores health data items 122, 124 on a per user basis, the key management system 200 may separately store a key pair 204, 210, 212 for each user. In implementations where the health data storage 120 stores the health data items 122, 124 on a per health data item basis, the key management system 200 may separately store a key pair 204, 210, 212 for each health data item 122, 124 uploaded to the health data storage 120.

The key splitter 214 may receive a key (e.g., a public key 206, a private key 208) or a key pair 204 and may split the key 206, 208 or key pair 204 into a plurality of key parts 216, 218, 220. As depicted, the key management system 200 also includes a plurality of key holders 224, 228, 232. In certain implementations, the key splitter 214 may split the key 206, 208 or key pair 204 (e.g., the private key 208) into the same number of key parts 216, 218, 220 as there are key holders 224, 228, 232. For example, as depicted, the key management system 200 includes three key holders 224, 228, 232. Accordingly, the key splitter 214 as depicted may split the private key 208 or key pair 204 into three key parts 216, 218, 220 (e.g., into three equally-sized parts of the private key 208). In certain implementations, the key splitter 214 is implemented as a software module of the user portal 110, e.g., on a user device 108. In such implementations, the key splitter 214 may receive the key from another software or hardware module of the user portal 110, e.g., a memory of the user device 108. When splitting the private key 208 or key pair 204 into the key parts 216, 218, 220, the key splitter 214 may also generate a checksum for each key part 216, 218, 220, which may be subsequently used to confirm the validity of each key part.

The key holders 224, 220, 232 may each receive a single key part 216, 218, 220 and may encrypt the received key parts 216, 218, 220 into a corresponding encrypted key part 226, 230, 234. For example, the key part 216 may be encrypted using a private key of the key holder 224 to create the encrypted key part 226. The key holder 228 may similarly encrypt the key part 218 to create the encrypted key part 230 and the key holder 232 may encrypt the key part 220 to form the encrypted key part 234. The key holders 224, 228, 232 may store the encrypted key parts 226, 230, 234 for later use in downloading, accessing, and validating health data items 122, 124 corresponding to the received key 206, 208 or key pair 204. The number of key holders 224, 228, 232 may be dynamically adjusted based on the needs of the key management system 200 by the system 100. For example, accurate decryption of the encrypted key parts 226, 230, 234 may still be possible if additional key holders 224, 228, 232 are added to the key management system 200 (e.g., 2 key holders are added in addition to the key holders 224, 228, 232 for a total of five key holders). The key parts 216, 218, 220 may be encrypted prior to transmission from the key splitter 214 and the key holders 224, 228, 232. For example, the key parts 216, 218, 220 may be transmitted to the key holders 224, 228, 232 using a secret box. The secret box may be implemented as, e.g., a JavaScript Object Notation (JSON) file that includes the respective encrypted key part 226, 230, 234 and a checksum generated by the key splitter, as described above.

The validators 236, 230, 240 may be configured to perform a download validation operation 150, 156 for a health data item 122, 124 being accessed by a customer (e.g., via a customer portal 106). Prior to performing the download validation operation 150, 156, one of the validators 236, 238, 240 may request the encrypted key parts 226, 230, 234 from the key holders 224, 228, 232. Each key holder 224, 228, 232 may decrypt a corresponding key part 226, 230, 234 using a private key associated with the key holder 224, 228, 232. The key holders 224, 228, 232 may then provide the decrypted key parts to the validator 236, 238, 240. The validator 236, 238, 240 may then reconstruct the key 206, 208 or key pair 204 from the decrypted key parts 216, 218, 220. The validator 236, 238, 240 may then use the reconstructed key 206, 28 or key pair 204 to decrypt the health data item 122, 124 and may then perform the download validation operation 150, 156 on the decrypted health data item 122, 124.

After validating the file, a hash may be generated, e.g., by the customer portal or by at least one of the key holders 224, 228, 232. The hash may then be divided into multiple parts, e.g., one part for each key holder 224, 228, 232 storing an encrypted key part 225, 230, 234 for the health data item 122, 124 being accessed. Each corresponding key holder 224, 228, 232 may receive a part of the hash and may use the parts to encrypt the respective key part 216, 218, 220. For example, each key holder 224, 228, 232 may use its corresponding part of the hash as a password under the Advanced Encryption Standard-128 (AES-128) encryption protocol in combination with encrypting the key part 216, 218, 220 using a public key corresponding to each key holder 224, 228, 232. After being re-encrypted by the key holder 224, 228, 232, the re-encrypted key parts may then be transferred to a storage while the customer finishes the purchasing process.

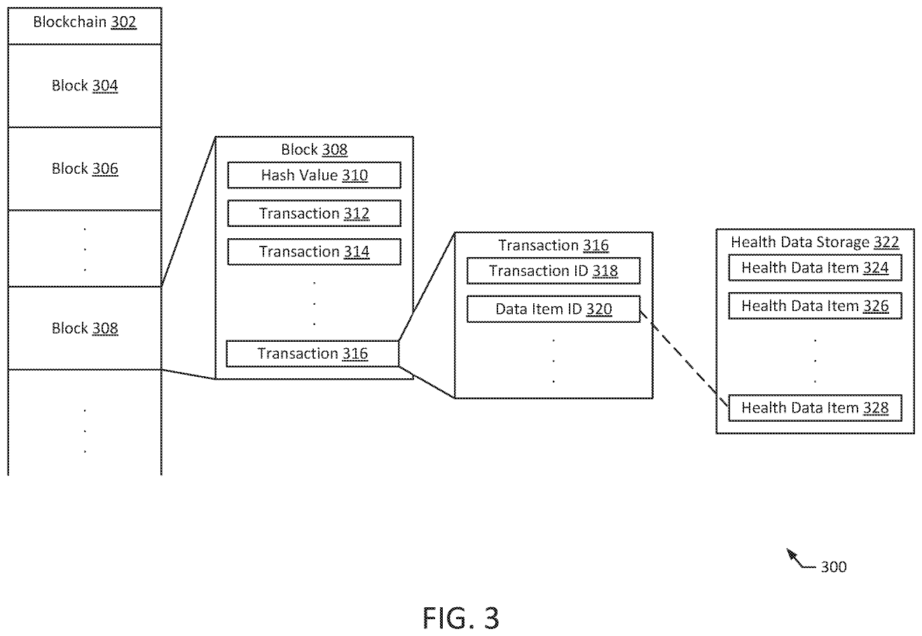

FIG. 3 depicts a block diagram of a blockchain-based health data storage system 300 according to an exemplary embodiment of the present disclosure. The system 300 may be used to implement one or more portions of the system 100, such as the blockchain 112 and the health data storage 120. The system 300 includes a blockchain 302 and a health data storage 322.

As discussed above, the blockchain 302 may be implemented by a plurality of nodes 114, 116, 118 and may be configured to store a plurality of transactions relating to health data items 324, 326, 328 stored on the health data storage 322. For example, the blockchain 302 may be configured to store data, permission assignment transactions, upload validation transactions, and download validation transactions. By storing these transactions, the blockchain 302 may act as a highly distributed storage system (HDSS). For example, the distributed nature of the nodes 114, 116, 118 implementing the blockchain 302 may improve the reliability and speed to access data stored on the blockchain 302, as compared against more conventional data storage solutions.

As depicted, the blockchain 302 may be configured to include a plurality of blocks 304, 306, 308. The blocks 304, 306, 308 may be appended one after another to create a continuous ledger. For example, the blocks may be appended to the blockchain 302 by the nodes 114, 116, 118 at regular intervals. As depicted, block 308 includes a plurality of transactions 312, 314, 316. These transactions may include data upload transactions, permission assignment transactions, upload validation transactions, and download validation transactions. The block 308 may also includes a hash value 310, which may be calculated according to one or more consensus requirements.

In one implementation, the consensus requirements may include a byzantine fault tolerant (BFT) consensus. For example, one of the nodes 114 may propose a block 308 of transactions 312, 314, 316 for inclusion on the blockchain 302. The other nodes 116, 118 may then review the proposed block 308 and may prevote to approve the block 308 if the other nodes 116, 118 have identical copies of all of the transactions 312, 314, 316 included in the block 308. If the proposed block 308 receives a supermajority of prevote approvals from the other noted 116, 118, the node 114 may then transmit a hash value 310 indicating a new state of the blockchain 302 (e.g., the state of the blockchain 302 including the block 308) for approval by the other nodes 116, 118. If a supermajority of the other nodes 116, 118 approve, the node 114 may then add the block 308 to the blockchain 302, along with the hash value 310.

In another implementation, the consensus requirements may include a proof of work (POW) consensus. For example, the nodes 114, 116, 118 may work to determine a hash value 310 that meets one or more POW requirements (e.g., a hash value 310 that, when hashed with the transactions 312, 314, 316 of the block 308 results in a number with a predetermined number of leading zeroes). After calculating the hash value 310 for the block 308, the calculating node 114 may transmit the block 308 including the hash value 310 to the other nodes 116, 118 implementing the blockchain 112. The other nodes 116, 118 may then verify the accuracy of the hash value 310 determined by the calculating node 114, 116, 118. After determining that the hash value 310 is accurate, a consensus may thus be reached by the nodes 114, 116, 118 and the block 308, including the hash value 310 and the transactions 312, 314, 316 may be appended to the blockchain 302.

By ensuring the hash value 310 meets one or more consensus requirements, the system 300 may ensure that the blocks 304, 306, 308 of the blockchain 302 are cryptographically linked and therefore difficult or impossible to tamper with. The consensus conditions may also help guarantee that the blockchain 302 remains in the same state for every node 114, 116, 118 and cannot be altered without the agreement of the other nodes, which further helps protect against alteration of the transactions 312, 314, 316 stored on the blockchain 302.

Transactions 312, 314, 316 may be selected for inclusion within a block 308 according to one or more selection strategies, including a priority-based selection, a first in first out (FIFO) selection, or an auction-based selection method. After selecting the transactions 312, 314, 316 for inclusion within the block 308, the nodes 114, 116, 118 may then proceed with calculating the hash value 310. After the block 308 is appended to the blockchain 302, the nodes 114, 116, 118 may proceed with selecting transactions for inclusion within a subsequent block and calculating the corresponding hash value 310. In this way, the blockchain 302 may store a plurality of transactions reflecting operations involving a health data storage 322 or health data items 324, 326, 328 without having to trust the accuracy or authenticity of any single node 114, 116, 118. Although the hash value 310 and transactions 312314, 316 are only depicted for the block 308, the other blocks 304, 306 of the blockchain 302 may include similar items.

In certain implementations, the blockchain 302 may be implemented using the Exonum framework. The Exonum framework may provide several benefits. First, the Exonum framework may enable a service-oriented architecture that may make it easier to deploy and adjust services that utilize the blockchain 302 (e.g., the validators 144, 140, 152, 236, 238, 240, the user portal 110, and the customer portal 106). Second, the Exonum framework may improve access to the blockchain by auditors to help ensure reliability for the system 100. Third, the Exonum framework may provide improved transaction throughput, which may allow for, e.g., greater logical complexity and greater volume processing by the system 100.

Further, the blockchain 302 may periodically store an anchored state of the blockchain 302 at that time on another, more broadly-used blockchain system (e.g., Bitcoin, Ethereum). In certain implementations, the anchored state of the blockchain may include a hashed value of the blockchain 302 at that time.

The transaction 316 includes a transaction ID 318 and a data item ID 320. The transaction ID 318 may identify the transaction 316 and may include both a transaction ID number identifying the specific transaction 316 and a transaction type (e.g., whether the transaction 316 is a data upload transaction, a permission assignment transaction, and upload validation transaction, or a download validation transaction). The data item ID 320 may identify the health data item 328 corresponding to the transaction 316 within the health data storage 322. For example, each health data item 324, 326, 328 within the health data storage 322 may include a specific identifier (e.g., an ID number). The data item ID 320 may thus store the specific identifier associated with the health data item 328 to which the transaction 316 applies. Although only transaction 316 as depicted in detail, transactions 312, 314 may similarly include transaction ID 318 and the data item ID 320. Additionally, as discussed in greater detail below, the transaction 316 may include additional details beyond the transaction ID 318 in the data item ID 320.

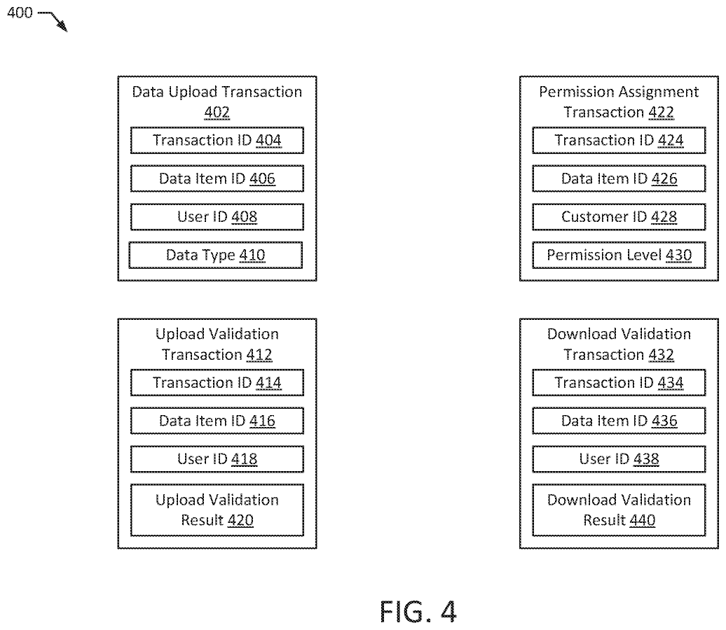

FIG. 4 depicts a plurality of transactions 400 according to exemplary embodiments of the present disclosure. The transactions 400 include a data upload transaction 402, a permission assignment transaction 422, and upload validation transaction 412, and a download validation transaction 432. Each of the transactions 402, 422, 412, 432 respectively include transaction IDs 404, 424, 414, 434 similar to the transaction ID 300 and the team discussed above in connection with FIG. 3. Similarly, the transactions 402, 422, 412, 432 respectively include data item IDs 406, 426, 416, 436 similar to the data item ID 320 discussed above in connection with FIG. 3.

The data upload transaction 402 may reflect a data upload operation uploading a health data item 122, 124, 324, 326, 328 to a health data storage 120, 322, e.g., via a user portal 110. For example, the data upload transaction 400 and to may reflect a data upload operation where a user has uploaded the health data item 122, 124, 324, 326, 328 associated with the data item ID 406 on the health data storage 120, 322. The data upload transaction 402 may include a user ID 408 and a data type 410. The user ID 408 may include an indication of the user who uploaded the health data item 122, 124, 324, 326, 328 to the health data storage 120, 322. In certain implementations, the user associated with the user ID 408 may be an individual associated with the health data item 122, 124, 324, 326, 328. For example, the health data item 122, 124, 324, 326, 328 may indicate one or more health conditions or health readings of a user. In other implementations, the user associated with the user ID 408 that uploads the health data item 122, 124, 324, 326, 328 may not be the same person as the individual corresponding to the health data item 122, 124, 324, 326, 328. For example, the user ID 408 may be associated with a healthcare provider uploading an individual's health data items 122, 124, 324, 326, 328 on behalf of the individual. Similar users may include, e.g., healthcare application providers, trusted individuals, and healthcare device manufacturers.

The data type 410 may indicate the type of data for the uploaded health data item 122, 124, 324, 326, 328 associated with the data item ID 406. For example, the data item 410 may indicate that the health data item is, e.g., hair composition, an image of an eye, and image of acne or another skin condition, an image of wrinkles, an image of the exterior of the individual's body, a tissue-specific transcriptome, a partial or complete genome sequence, an epigenome sequence, a clinical history, a blood text, MRI imaging data, CT imaging data, x-ray imaging data, SMP imaging data, a urine test, a social network feed, and social networking connection information. Additionally or alternatively, the data typed 410 may indicate formatting information for the health data item 122, 124, 324, 326, 328. For example, the data may be stored in one or more formats (e.g., CSV, image type, JSON, blob, tab delimited). The data type 410 may then be used to enable future processes that utilize the associated health data item 122, 124, 324, 326, 328 to account for the indicated formatting, which may improve processing accuracy and/or speed. For example, the system 100 (e.g., the customer portal 106 may include a parsing system configured to parse health data from health data items 122, 124 stored in a plurality of different data types. In such implementations, the parsing system may utilize the data type CDX to accurately parse the health data items 122, 124 according to the data type. In other implementations, the parsing system may be utilized during data upload, and may be configured to operate within the user portal 110.

The upload validation transaction 412 may be used to indicate the result of an upload validation operation 146, 154 performed by a validator 144, 152 on an uploaded data item, such as an uploaded data item associated with a data upload transaction 402. For example, a validator 144, 152 may download a health data item 122, 124, 324, 326, 328 associated with a data item ID 406 of a data upload transaction 402. The validator 144, 152 may then perform an upload validation operation 146,154 on the downloaded health data item 122, 124, 324, 326, 328 and may generate an upload validation result 420 associated with the result of the performed upload validation operation 146, 154. The validator 144, 152 may then store the upload validation results within the upload validation transaction 412. The upload validation transaction 412 also includes a user item ID 418, which may indicate the validator 144, 152 or a user associated with the validator 144, 152 responsible for performing the upload validation operation 146, 154.

The upload validation transaction 412 may then be stored on a blockchain 112, 300 to publicly indicate that the uploaded health data item 122, 124, 324, 326, 328 has been validated. In certain implementations, subsequent use of the uploaded health data item 122, 124, 324, 326, 328 (e.g., research-based efforts or diagnostic uses) may be prevented until the uploaded health data item 122, 124, 324, 326, 328 has been validated. This prevention may ensure that inaccurate, incompatible, or falsified health data items 122, 124, 324, 326, 328 are not uploaded to the health data storage 120, 322 and are not incorrectly used for subsequent medical purposes.

The permission assignment transaction 422 may be configured to indicate the assignment of access permissions to one or more customers (e.g., research institutions, hospitals, or other medical actors). The permission assignment transaction 422 includes a transaction ID 424 identifying the specific transaction, as well as a data item ID 426 identifying, e.g., the health data item 122, 124, 324, 326, 328 for which access permissions are being assigned. In certain implementations, the permission assignment transaction 422 may correspond to more than one health data item 122, 124, 324, 326, 328. In such instances, the data item ID 426 may include identifiers for a plurality of associated health data items 122, 124, 324, 326, 328.

The permission assignment transaction 422 further includes a customer ID 428. The customer ID 428 may be used to identify the associated customer of the system for which access permissions for the health data items 122, 124, 324, 326, 328 are being assigned. For example, the customer ID 428 may indicate an ID associated with a user name for the customer being granted permissions by the permission assignment transaction 422. In certain implementations, the customer ID may be similarly formatted to the user IDs 408, 418 stored in the data upload transaction 402 and the upload validation transaction 412. For example, an individual may have a single user account that is used both to upload health data items 122, 124, 324, 326, 328 corresponding to the individual and to purchase or request to access health data items 122, 124, 324, 326, 328 uploaded by other users (e.g., for research purposes). Accordingly, the same identifier may be used as a user ID 408, 418 in the data upload transaction 402 and the upload validation transaction 412 and as a customer ID 428 for the permission assignment transaction 422.

The permission assignment transaction 422 also includes a permission level 430 that indicates the level of access granted to the individual or individuals associated with the customer ID 428 for the health data items 122, 124, 324, 326, 328 associated with the data item ID 426. For example the permission level 430 may indicate that the customer associated with the customer ID 428 permission to have read-only access to a health data item 122, 124, 324, 326, 328, download a copy of the raw data included in health data item 122, 124, 324, 326, 328, and/or to have access to data resulting from calculations performed on the health data item 122, 124, 324, 326, 328.

The download validation transaction 432 may be used to indicate the result of a download validation operation 150, 158 performed by a validator 148, 152. The download validation operation 150, 156 may be performed responsive to, e.g., a request to access a health data item 122, 124, 324, 326, 328. For example, the download validation operation 150, 156 may be performed to ensure that the health data item 122, 124, 324, 326, 328 has not been corrupted or otherwise interfered with. In certain implementations, a validator 144, 152 may download a health data item 122, 124, 324, 326, 328 associated with the data item ID 406, e.g., in response to a data access request. The validator 144, 152 may then perform a download validation operation 146, 154 on the downloaded health data item 122, 124, 324, 326, 328 and may generate a download validation result 420 associated with the result of the performed upload validation operation 146, 154. The validator 144, 152 may then store the upload validation result 420 within the upload validation transaction 412. The upload validation transaction 412 also includes a user item ID 438, which may indicate the validator 144, 152 (e.g., a user associated with the validator 144, 152) responsible for performing the upload validation operation 146, 154.

In certain implementations, the transactions 402, 412, 422,432 may include additional elements. The transactions 402, 412, 422, 432 may also exclude certain elements depending on, e.g., the configuration of the blockchain 302 and the validators 144, 140, 152.

### Method Descriptions

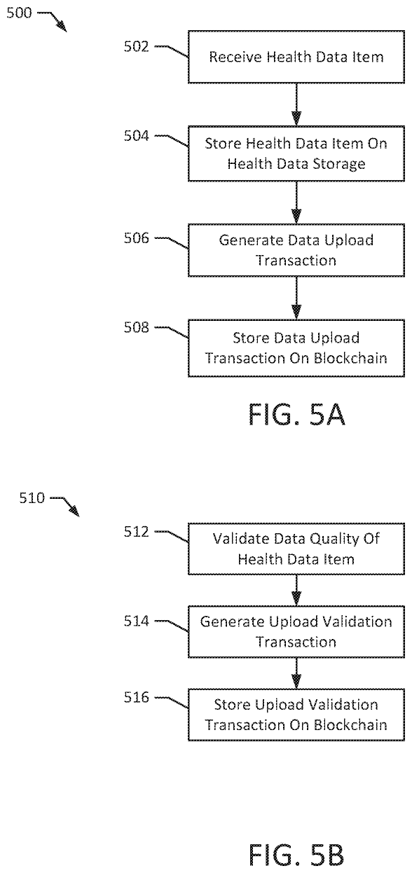

FIG. 5A depicts a flowchart of a method 500 for data upload according to exemplary embodiments of the present disclosure. The method 500 may be performed to upload a health data item 122, 124, 324, 326, 328 to a health data storage 120, 322. For example, a user portal 110 may be configured to perform the method 500 to upload a health data item 122, 124, 324, 326, 328 from a user device 108 to a health data storage 120, 322 and to store a corresponding data upload transaction 402 on a blockchain 112, 302 reflecting the health data item 122, 124, 324, 326, 328 uploaded to the health data storage 120, 322.

The method 500 may be implemented on at least one computer system. For example, one or more steps of the method 500 may be implemented by the user portal 110, user device 108, and health data storage 120. Although the examples below are described with reference to the flowchart illustrated in FIG. 5A, many other methods of performing the acts associated with FIG. 5A may be used. For example, the order of some of the blocks may be changed, certain blocks may be combined with other blocks, one or more of the blocks may be repeated, and some of the blocks described may be optional.

The method 500 may begin with the system 100 receiving a health data item 122, 124, 324, 326, 328 (block 502). The health data item may be received from a user portal 110. For example, a user device 108 implementing a user portal 110 may transmit the health data item to the health data storage 120, 322 via the network 102.

The health data item 122, 124, 324, 326, 328 may then be stored on the health data storage 120, 322 (block 504). For example, the health data storage 120, 322 may be configured to store health data items 122, 124, 324, 326, 328 in one or more data structures. In certain implementations, the health data storage 120, 322 may be configured to store health data items 122, 124, 324, 326, 328 with similar data types 410 in separate tables of the health data storage 120, 322. When storing the health data item on the health data storage 120, 322, the system 100 (e.g., a user portal 110) may store an indication of the location of the health data item 122, 124, 324, 326, 328 stored within the health data storage 120, 322.

The user portal 110 may then generate a data upload transaction 402 (block 506). As described above, the data upload transaction 402 may include a transaction ID 404, data item ID 406, user ID 408, and a data type 410. In creating the user portal 110, the user portal 110 may store the location of the health data item 122, 124, 324, 326, 328 in the health data storage 120, 322 and may use that stored location to create the data item ID 406. Additionally, the user portal 110 may determine a data type 410 for the uploaded health data item 122, 124, 324, 326, 328 for inclusion within the data type 410. For example, the health data item 122, 124, 324, 326, 328 may include an indicator of the type of health data stored within the health data item 122, 124, 324, 326, 328 and the user portal 110 may extract the indicator and convert it into the data type 410. Alternatively, the user portal 110 may ask the user for a data type of the uploaded health data item 122, 124, 324, 326, 328 and may assign the data type provided by the user as the data type 410 of the data upload transaction 402. Additionally, in generating the user ID 408, the user portal 110 may use user account information associated with the user. For example, the user ID 408 may include the user account name of the user who uploaded the health data item 122, 124, 324, 326, 328, or a user with whom the health data item is associated (e.g., a username associated with the individual from whom the health data item 122, 124, 324, 326, 328 originated). As discussed above, the user ID 408 may also store information associated with a user that uploaded the health data item 122, 124, 324, 326, 328 on behalf of another individual, e.g., a patient. In other implementations (e.g., where data privacy is required), the user portal 110 may convert the user name of the user into an anonymized user identifier, such as a public key of the user, or a public, encrypted identifier. In certain implementations, applicable regulations may require that only anonymized user identifiers be included in the data upload transaction 402. As depicted in FIG. 4, the transaction ID 404, data item ID 406, user ID 408, and data type 410 may then be collected into a single data structure to form the data upload transaction 402.

In certain implementations, the data upload transaction 402 may be generated by a customer portal 106. For example, the customer portal 106 may generate a data upload transaction 400 if health data items 122, 124, 324, 326, 328 generated by a customer device 104 or a customer (e.g., health data items 122, 124, 324, 326, 328 resulting from research using data stored within the system 100).

In further embodiments, the system 100 may score of the uploaded health data item 122, 124, 324, 326, 328 prior to storing it on the health data storage 120, 322 or prior to creating the data upload transaction 402. For example, the user portal 110 may include one or more data scoring mechanisms (e.g., neural network libraries, machine learning models, heuristic scoring techniques). The data scoring mechanisms may be created by other users of the system 100, such as customers of the system. The data scoring mechanisms may analyze the uploaded health data item 122, 124, 324, 326, 328 and may provide a quality score and/or analysis of the uploaded health data item 122, 124, 324, 326, 320. In certain implementations, the quality score and/or analysis may be included within the data upload transaction 402.

The user portal 110 may then store the data upload transaction 402 on the blockchain 122, 302 (block 506). For example, the user portal 110 may broadcast the data upload transaction 402 to the nodes 114, 116, 118 implementing the blockchain 112, 302, which may then store the data upload transaction 402 in a block 304, 306, 308 of the blockchain 122, 302 using a consensus algorithm, as discussed above. The nodes 114, 116, 118 may then group the data upload transaction 402 with other transactions (e.g., upload validation transactions 412, permission assignment transactions, download validation transactions 432, and other data upload transactions 402) to form a block 308. After verifying that the block 308 complies with the requirements of the system, the block 308 may then be appended to the end of the blockchain 122, 302. In this way, the blockchain 112, 302 may include a record of the uploaded health data item 122, 124, 324, 326, 328, along with records of all of the health data items 122, 124, 324, 326, 328 stored in the health data storage 120, 322.

Prior to grouping the data upload transaction 402, the nodes 114, 116, 118 may verify that the customer or user that created the data upload transaction 402 is authorized to use the system 100. For example, the data upload transaction 402 may include a public key of the customer or user that created the data upload transaction 402. This public key may correspond to a wallet of a user that was authorized via a wallet creation transaction added to the blockchain 112, 302. In such implementations, the nodes 114, 116, 118 may verify the customer or user by searching for a wallet creation transaction corresponding to the public key within the blockchain 112, 302. If the customer or user is validated, the node 114, 116, 118 may proceed with grouping the data upload transaction 402 with other transactions. If the customer or user validation fails, the node 114, 116, 118 may mark the health data item 122, 124, 324, 326, 328 associated with the created data upload transaction 402 as “lost” or “invalid.”

FIG. 5B depicts a flowchart of a method 510 for uploading health data items 122, 124, 324, 326, 328 according to an exemplary embodiment of the present disclosure. The method 510 may be performed to validate one or more health data items 122, 124, 324, 326, 328 uploaded to a health data storage 120, 322. For example, the method 510 may be performed by the system 100 to validate a health data item 122, 124, 324, 326, 328 that has been uploaded to a health data storage 120, 322 and reflected in a data upload transaction 422 of the blockchain 112, 302.

The method 510 may be implemented on at least one computer system. For example, one or more steps of the method 510 may be implemented by the validator 144, 152. Although the examples below are described with reference to the flowchart illustrated in FIG. 5B, many other methods of performing the acts associated with FIG. 5B may be used. For example, the order of some of the blocks may be changed, certain blocks may be combined with other blocks, one or more of the blocks may be repeated, and some of the blocks described may be optional.

The method 510 may begin with a validator 144, 152 validating a data quality of a health data item 122, 124, 324, 326, 328 (block 512). In certain implementations, the validator 144, 152 may be configured to validate all data uploaded to the health data storage 120, 322 and reflected in a data upload transaction 402 on the blockchain 112. For example, when a validator 144, 152 detects a new data upload transaction on the blockchain 112, 302, the validator 144, 152 may download that health data item 122, 124, 324, 326, 328 from the health data storage 120, 322 and may then validate a data quality of the health data item 122, 124, 324, 326, 328. In other implementations, the validator 144, 152 may be configured to validate only a subset of the data uploaded to the health data storage 120, 322 (e.g., all data of a certain type, all data from patients located in a certain area, all data uploaded by a particular user or users). In such implementations, the validator 144, 152 may then download and validate the data quality of all health data items 122, 124, 324, 326, 328 that meet the prescribed criteria for the validator 144, 152.

In validating a data quality of the health data item 122, 124, 324, 326, 328, the validator 144, 152 may be configured to perform one or more upload validation operations 146, 154. The upload validation operations 146, 154 may be specialized operations developed by an organization in charge of implementing the validator 144, 152. For example, different institutions may elect to implement validators 144, 152, 148 of the system 100 to validate different types of health data items 122, 124, 324, 326, 328. In these cases, the institutions implementing each validator 144, 152 may independently develop and maintain the upload validation operations 146, 154 in exchange for incentive mechanisms such as cryptocurrency. For example, one institution may specialize in validating cancer-related health care screening data. The institution may then develop upload validation operations 146, 154 for health data items 122, 124, 324, 326, 328 (e.g., imaging results) originating from patients with our suspected of having cancer. In certain implementations, the upload validation operation may be implemented as a machine learning model trained on specific types of health data items 122, 124, 324, 326, 328. For example, the institution implementing the validator 144, 152 may have previously purchased access to other health data items stored in the health data storage 120, 322. The institution may then train the machine learning model implementing the upload validation operation 146, 154 to accurately validate the data quality of the health data item 122, 124, 324, 326, 328. For example, the upload validation operation 146, 154 may be trained to make sure that the data included in the health data item 122, 124, 324, 326, 328 (e.g., blood test readings, imaging results) conform to one or more data quality requirements, based on the specialized area of focus of the validator 144, 152.

The specific data quality validated by the upload validation operation 146, 154 may differ depending on the type of data. For example, the validator 144, 152 may be trained to validate one or more aspects of the data that are likely to be corrupted and/or incorrect based on previous samples of similar data types. For example, the validator 144, 152 may be configured to confirm a compatible file format or data storage format of the health data item 122, 124, 324, 326, 328 to ensure that the health data item 122, 124, 324, 326, 328 is compatible with typical usage by researchers or other customers of the system 100. In other implementations, the upload validation operation 146, 154 may be configured to detect and/or correct one or more aspects of health data items that are typically prone to error. For example, an uploaded health data item 122, 124, 324, 326, 328 may indicate (e.g., in a data type 410 of the data upload transaction 402 that the uploaded health data item 122, 124, 324, 326, 328 corresponds to a patient with a particular kind of cancer (e.g., lung cancer). The uploaded health data item 122, 124, 324, 326, 328 may include one or more images reflecting, e.g., a CT scan, MRI scan, or other imaging scan, performed on the associated individual. The upload validation operation 146, 154 may be configured to analyze such images to validate the assertion (e.g., by the data type 410) that the images reflect a patient with lung cancer. For example, the upload validation operation 146, 154 may include a machine learning model trained to analyze such images to determine a likelihood that the images depict a patient with lung cancer. Similar validators may be possible for other health conditions, such as other cancers or other conditions based on imaging results. Some implementations are possible based on non-imaging health data items 122, 124, 324, 326, 328. For example, an upload validation operation 146, 154 may be configured to validate an assertion in a data type 410 that health data items 122, 124, 324, 326, 328 correspond to patients with one or more blood conditions (e.g., anemia) based on one or more blood test readings included within the health data item or items 122, 124, 324, 326, 328 corresponding to the patient uploaded previously to the health data storage 120, 322. In such implementations, the upload validation operation 146, 154 may be able to validate a data quality, such as the data quality of the data type 410 asserting specific medical condition. Such machine learning models may further validate data qualities such as accuracy of one or more sensor readings. For example, a sensor reading uploaded in a health data item 122, 124, 324, 326, 328 for a user that differs greatly from sensor readings uploaded in previous health data items may be rejected by the upload validation operation 146, 154 as invalid if the difference exceeds a certain threshold, as determined by the upload validation operation 146, 154.

Because the validators 144, 152 may be implemented by institutions other than the institution implementing, e.g., the blockchain 112, 302 the health data storage 120, 322, the customer portal 106, and the user portal 110, the system 100 may be better equipped to handle health data items 122, 124, 324, 326, 328 corresponding to new conditions and may be better able to leverage expertise in many more domains, as experts in new domains (e.g., new health conditions or novel methods of diagnosis/treatment) may implement their own validators 144, 152 to validate uploaded health data items 122, 124, 324, 326, 328 in exchange for incentive mechanisms, such as utility tokens and cryptocurrency.

The validator 144, 152 may then generate and upload validation transaction 412 (block 514). The upload validation transaction 412 may include a transaction ID 414 indicating a unique identifier for the specific upload validation transaction 412 being generated. The upload validation transaction may also include a data item ID 416. For example, the validator 144, 152 may retrieve data item ID 416 from the data item ID 406 for the data upload transaction 400 and corresponding to the health data item 122, 124, 324, 326, 328 being validated. The data item ID 406 may be used to both retrieve the health data item 122, 124, 324, 326, 328 from the health data storage 120, 322 and to generate the data item ID 416 included within the upload validation transaction 412. The upload validation transaction 412 may also include a user ID 418, which may correspond to a user ID of the entity implementing the validator 144, 152 or a user ID 418 corresponding to the validator 142, 152 itself. For example, one institution may implement a plurality of validators 142, 152 (e.g., a separate validator with a separate upload validation operation 146, 154 configured to analyze health data items 122, 124, 324, 326, 328 corresponding to a plurality of individual medical conditions). In such implementations, that institution may use a separate user ID for each validator 140, 144, 152, 236, 238, 240 validating health data items 122, 124, 324, 326, 328 corresponding to each health data condition, or may use a single user ID for a plurality of the health conditions (e.g., all of the validators 140, 144, 152, 236, 238, 240 implemented by the institution).

The upload validation transaction 412 may also include an upload validation result 420. The upload validation result 420 may include an indication that the associated health data item 122, 124, 324, 326, 328 has been validated. For example, the upload validation result 420 may be implemented as a binary indicator of a pass or a fail result of the upload validation operation 146, 154. In other implementations the upload validation result 420 may include additional details about the upload validation operation 146, 154 performed on the uploaded health data item. For example, if the upload validation operation 146, 154 detects one or more errors in the formatting of the health data item 122, 124, 324, 326, 328, the validator 144, 152 may take action to correct these formatting errors, and the corrective steps may be recorded in the upload validation result 420. In another example, the upload validation result 420 may include a specific identifier as to the detected error in the health data item 122, 124, 324, 326, 328. For example, the upload validation result 420 may identify one or more suspect portions of an imaging result asserted to originate from a patient with a given cancer, such as portions of the imaging results that generally indicate that the patient does not have cancer (e.g., missing areas of varying density suggesting tumor growth).

The validator 144, 152 may then store the upload validation transaction 412 on the blockchain 112, 302 (block 516). For example, the validator 144, 152 may broadcast the data upload transaction 402 to the nodes 114, 116, 118 implementing the blockchain 112, 302, which may then store the upload validation transaction 412 in a block 304, 306, 308 of the blockchain 122, 302 using a consensus algorithm, as discussed above. In certain implementations, the system 100 and the nodes 114, 116, 118 may not distinguish between the procedure used to store data upload transactions 402 and upload validation transactions 412. For example, the system 100 may be configured to store transactions on the blockchain hundred and 12, 302 on a first in first out (FIFO) system, where the transactions 402, 412 are processed in the same order in which they are received. In other implementations, the system 100 may be configured to prioritize data upload transactions 402 or upload validation transactions 412. For example, the system 100 may prioritize storage of health data items 122, 124, 324, 326, 328 into the health data storage 120, 322. In such implementations, the system 100 may process data upload transactions 400 and to prior to processing upload validation transactions 412.

By performing the steps of the method 510, the validators 144, 152 may be able to validate uploaded health data items 122, 124, 324, 326, 328 and ensure that the quality of the health data items 122, 124, 324, 326, 328 uploaded to the system 100 is of a high enough quality to ensure successful use of the health data items 122, 124, 324, 326, 328 in future research efforts. In this way, the overall value of the health data stored on system may be insulated from decay due to users uploading suspect or inaccurately-determined health data items.

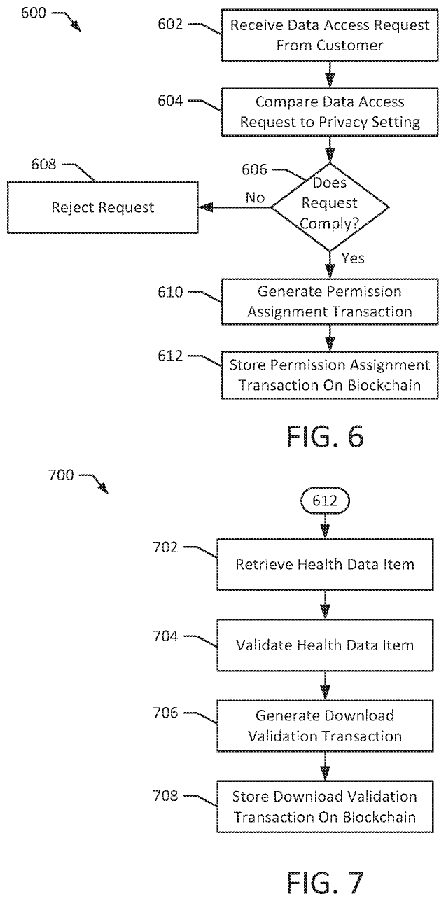

FIG. 6 depicts a flow chart of a method 600 for providing data access according to an exemplary embodiment of the present disclosure. The method 600 may be performed by one or more components of a system 100, such as by the customer portal 106, the blockchain 112, and the health data storage 120. Performing the steps of the method 600 may enable customers such as hospitals, research institutions, and other medical research parties to purchase or otherwise request access to one or more health data items 122, 124, 324, 326, 328 stored on the health data storage 120, 322. For example, the research institution researching a medical condition (e.g., pancreatic cancer) may require many health data items 122, 124, 324, 326, 328 from patients diagnosed as having the researched medical condition. Accordingly, the research institution may request access or purchase access to the corresponding health data items to, e.g., better train and associated machine learning model or to perform other types of medical research.

The method 600 may be implemented on at least one computer system. For example, one or more steps of the method 600 may be implemented by the customer portal 106, the blockchain 112, and the health data storage 120. Although the examples below are described with reference to the flowchart illustrated in FIG. 6, many other methods of performing the acts associated with FIG. 6 may be used. For example, the order of some of the blocks may be changed, certain blocks may be combined with other blocks, one or more of the blocks may be repeated, and some of the blocks described may be optional.

The method 600 may begin with the system 100 receiving a data access request from a customer (block 602). The data access request may specify a particular health data item for which access is requested. For example, the data access request may include a specific data item ID 400 and to corresponding to a particular health data item #on the health data storage 120, 322. In other instances, the data access request may specify particular types of health data items 122, 124, 324, 326, 328 for which access is requested. For example, in instances where the customer is researching a particular disease or medical condition, the data access request may include one or more criteria health data items for which access is requested. For example, the data access request may specify that axis is only required for health data items 122, 124, 324, 326, 328 originating from patients diagnosed with the researched medical condition. In other instances, the research institution may be researching a particular segment of the population and the data access request may include criteria corresponding to the researched population segment. For example, the data access request may specify an age, gender, income, or other demographic information for the patient corresponding to the health data item 122, 124, 324, 326, 328.

In certain implementations, the system 100 may provide for a purchasing mechanism that enables the customer to exchange one or more currencies (e.g., fiat currencies, crypto currencies, blockchain-based utility tokens) for access to health data items. For example, the exchanged currencies may be split between an operator of the system 100 and the user associated with access to health data items. In other implementations, the user associated with the health data item 122, 124, 324, 326, 328 may receive all of the exchanged currency. In such an instance, the data access request received from the customer may include a amount of currency that the customer is willing to exchange for access to the health data item 122, 124, 324, 326, 328. Alternatively or additionally, the data access request may also include different amounts of exchanged currencies in exchange for different levels of access to the health data items. For example, a customer may be willing to pay more for access to the raw data associated with a health data item 122, 124, 324, 326, 328, as opposed to only receiving access to calculations performed on that data. In other implementations, the data access request may also include a minimum or maximum number of health data items that the customer is willing to purchase access to.

The system may then compare the data access request with the privacy settings of one or more users (block 604) and may determine if the data access request complies with the privacy settings (block 606). For example, as discussed above, a user may set one or more privacy settings corresponding to their uploaded health data items 122, 124, 324, 326, 328. In addition, the user may also specify a minimum price to be accepted for access to their health data items 122, 124, 324, 326, 328. In these implementations, the system 100 may then compare the level of access requested in the data access request and/or the price included with the data access request to the settings of one or more users with health data items 122, 124, 324, 326, 328 that meet the conditions included within the data access request. For example, the data access request may be implemented as a smart contract that is run on the blockchain 112. Running the smart contract on the blockchain 112, 302 may initially return a plurality of health data items 122, 124, 324, 326, 328 that meet the conditions of the data access request. The system 100 or the smart contract may then compare the associated level of access to the health data items 122, 124, 324, 326, 328 requested within the data access request to the privacy settings of the users corresponding to the health data items 122, 124, 324, 326, 328 identified by running the smart contract on the blockchain 112, 302 and may remove the health data items 122, 124, 324, 326, 328 whose privacy settings conflict with the level of access requested in the data access request. For example, the data access request may only request access to the raw data included within the health data items 122, 124, 324, 326, 328, and one or more candidate health data items may only include privacy settings that allow for access to the results of calculations performed on the health data items. Accordingly such health data items 122, 124, 324, 326, 328 may be removed from the potential pool of health data items responsive to the data access request. Similarly, if a price included in the data access request does not meet the conditions set by the privacy settings of a user, the request may be deemed not to comply with the privacy settings of that health data item 122, 124, 324, 326, 328, and the health data item 122, 124, 324, 326, 328 may then be removed from the candidate pool.

As discussed above, if the data access request does not meet the privacy settings associated with one or more of the health data items 122, 124, 324, 326, 328, the data access request may be rejected (block 608). In implementations where the data access request is only requesting data to a single health data item 122, 124, 324, 326, 328, processing of the data access request may halt at block 608. In other implementations, where the data access request is requesting access to a plurality of health data items 122, 124, 324, 326, 328, rejecting the data access request for a particular health data item 122, 124, 324, 326, 328 may not end processing of the data access request, but may instead serve to remove the rejected health data item 122, 124, 324, 326, 328 from the candidate pool of health data items 122, 124, 324, 326, 328 responsive to the data access request.

If the data access request is determined to comply with the privacy settings for one or more health data items 122, 124, 324, 326, 328, the system 100 may then generate a permission assignment transaction 422 (block 610). As discussed above, similar to the data upload transaction 402 in the upload validation transaction 412, the permission assignment transaction 422 may include a transaction ID 424 identifying the specific transaction created at block 610. Relatedly, the permission assignment transaction 422 may have a data item ID 426 identifying the health data item or items 122, 124, 324, 326, 328 whose permissions are being assigned by the permission assignment transaction 422. For example, the data item ID 426 may identify the health data item or items 122, 124, 324, 326, 328 whose privacy settings the data access request received from the customer complied with a block 606. Thus, in certain implementations, the data item ID 426 may refer to more than one health data item 122, 124, 324, 326, 328.

The customer ID 428 may indicate a username or user identification number for the customer that submitted the data access request. Similar to the user ID 408, 418, the customer ID 428 may either refer directly to the customer or may be anonymized to refer indirectly to the customer. The permission level 430 may indicate the level of access being granted to the health data items 122, 124, 324, 326, 328 corresponding to the data item ID 426. For example, the permission level 430 may indicate that the customer associated with the customer ID 428 has read-only access to the health data items corresponding to the data item ID 426. In another example, the permission level 430 may indicate that the customer associated with the customer ID 428 has access only to the results of calculations performed on the health data items 122, 124, 324, 326, 328 corresponding to the data item ID 426. In implementations where the data item ID 426 identifies more than one health data item 122, 124, 324, 326, 328, the permission level 430 may similarly include a separate indicated access level for each health data item corresponding to the data item ID 426. In implementations where the data access request is a smart contract including one or more parameters for the requested health data items 122, 124, 324, 326, 328, certain health data items may have privacy settings that differ from one another, while still complying with the parameters of the data access request. For example, a first group of identified health data items 122, 124, 324, 326, 328 may allow read-only access, while a second group of health data items 122, 124, 324, 326, 328 may include privacy settings allowing for the customer to download an anonymized copy of the health data item 122, 124, 324, 326, 328. In such an example, the permission level 430 may separately include a “read only” permission level for the first group of health data items 122, 124, 324, 326, 328 (e.g., individually or as a group) and may separately indicate an “anonymous download” permission level for the second group of health data items 122, 124, 324, 326, 328 (e.g., individually or as a group).

The system 100 may then store the permission assignment transaction 422 on the blockchain 112, 302 (block 612). For example, the permission assignment transaction 422 may be broadcast to the nodes 114, 116, 118 implementing the blockchain 112, 302, which may then store the permission assignment transaction 422 in a block 304, 306, 308 of the blockchain 122, 302 using a consensus algorithm, as discussed above. As discussed above, in certain implementations, the nodes 114, 116, 118 may not differentiate between different types of transactions 402, 412, 422, 432, and may process the transactions 402, 412, 422, 432 on a FIFO basis. In other implementations, the nodes 114, 116, 118 may preferentially process certain types of transactions (e.g., data upload transactions 402) before other types of transactions.

By performing the steps of the method 600, the system 100 may be able to adjust the permission levels granted by users to their corresponding health data items 122, 124, 324, 326, 328 to grant and/or revoke access to one or more customers. Additionally, by storing the permission assignment transaction 422 on the blockchain 112, 302, the system 100 may ensure that the currently-assigned permission levels 430 are publicly auditable. Further, by incorporating a potential pricing/compensation factor within the user's privacy settings (e.g., at blocks 604, 606), the system 100 is able to compensate users for uploading health data items 122, 124, 324, 326, 328 that are useful in medical research, and therefore more likely to be purchased by customers via data access requests.

FIG. 7 depicts a flowchart of a method 700 for validating health data items #according to an exemplary embodiment of the present disclosure. In certain implementations, the method 700 may be performed by the system, e.g., validator 148, 152 to download and validate a health data item 122, 124, 324, 326, 328. For example, the health data item may be intended for use in medical research and may need to be validated, e.g., for accuracy and for potential data corruption prior to use in such research. For example, the method 700 may be performed in response to a data access request, such as the data access request received from a customer block 602 of the method 600. In such an example, the method 700 may proceed after completion of the method 600, e.g., after the block 612, as depicted in FIG. 7.

The method 700 may be implemented on at least one computer system. For example, one or more steps of the method 700 may be implemented by the validator 148, 152. Although the examples below are described with reference to the flowchart illustrated in FIG. 7, many other methods of performing the acts associated with FIG. 7 may be used. For example, the order of some of the blocks may be changed, certain blocks may be combined with other blocks, one or more of the blocks may be repeated, and some of the blocks described may be optional.

The method 700 may begin with the validator 148, 152 retrieving a health data item 122, 124, 324, 326, 328 (bock 702). The validator 148, 152 may retrieve the health data item in response to access permission being assigned to a customer to access that health data item. For example, the validator 140, 152 may detect a new permission assignment transaction 422 on the blockchain 112, 300 and to and may retrieve the health data item or items 122, 124, 324, 326, 328 referenced in the permission assignment transaction (e.g., in the data item ID 426) in response to detecting the permission assignment transaction 422. Alternatively, the validator 148, 152 may detect a separate download request from a customer for a health data item 122, 124, 324, 326, 328 that the customer is authorized to access and may retrieve the health data items 122, 124, 324, 326, 328 responsive to that download request.

The validator 148, 152 may then validate the retrieved health data item 122, 124, 324, 326, 328 (block 704). In certain implementations, the validator 148, 152 may perform a download validation operation 150, 156 on the retrieved health data item 122, 124, 324, 326, 328. For example, the download validation operation 150, 156 may include determining whether the requested health data item 122, 124, 324, 326, 328 is stored within the health data storage 120, 322. As with other data storage systems, it is possible that a health data item 122, 124, 324, 326, 328 previously uploaded to a health data storage 120, 322 is no longer stored on the health data storage 120, 322. For example, the health data item may have been corrupted, deleted, or moved without an updated record or transaction 312, 314, 316 being added to the blockchain 112, 302 due to system or user error. In such an implementation, the download validation operation 150, 156 may check for the requested health data item 122, 124, 324, 326, 328 at the location identified by, e.g., the data item ID 426 included in the permission assignment transaction 422 corresponding to the customer requesting the health data item 122, 124, 324, 326, 328, or at the location identified by the data item ID associated with the data upload transaction 402 of the health data item 122, 124, 324, 326, 328. If the validator 140, 152 determines that the requested health data item 122, 124, 324, 326, 328 is no longer stored at the location, the validator 148, 152 may determine that the accessed health data item is no longer stored at the health data storage 120, 322 and may upload a transaction 312, 314, 316, 402, 412, 422, 432 to the blockchain 112, 302 indicating that the requested health data item 122, 124, 324, 326, 328 is no longer available. Conversely, if the validator 140, 152 determines that the health data item is stored at the identified location, the validator 140, 152 may determine that the accessed health data item 122, 124, 324, 326, 328 is stored within the health data storage 120, 322. In certain implementations, the above-discussed steps may be performed while retrieving the health data item 122, 124, 324, 326, 328, e.g., during block 702.

In another example, the download validation operation 150, 156 may include decrypting the accessed health data item 122, 124, 324, 326, 328 and granting read-only access to the customer in accordance with a permission level 430 assigned to the customer, or a privacy setting of the user associated with the health data item 122, 124, 324, 326, 328. For example, as will be discussed further below, in certain implementations the health data items 122, 124, 324, 326, 328 may be encrypted before storage within the health data storage 120, 322. In such implementations, the encryption keys 206, 208 used to encrypt the health data item 122, 124, 324, 326, 328 before storage may be managed by a key management system 130, 200. In such implementations, the validator 148, 152 may retrieve the keys from the key management system 130, 200 and may decrypt the encrypted health data item and grant read-only access to the customer. For example, to grant read-only access, the validator may receive a computation to perform on the decrypted health data item (or plurality of health data items) 122, 124, 324, 326, 328. The validator may perform the designated operation on the decrypted health data item 122, 124, 324, 326, 328 and may then provide the result of the calculation to the customer for subsequent use (e.g., in research).

In a further example, where the permission level 430 indicates that the customer may download a copy of the health data item 122, 124, 324, 326, 328, the download validation operation 150, 156 may include downloading and decrypting the accessed health data item 122, 124, 324, 326, 328, as discussed above and re-encrypting the accessed health data item 122, 124, 324, 326, 328 with an encryption key associated with the customer. For example, to maintain privacy and security of the health data item 122, 124, 324, 326, 328 during transmission to the customer, the customer may provide, e.g., a public encryption key for use to encrypt the health data item prior to transmission. In performing the download validation operation 150, 156, the validator 148, 152 may then encrypt the health data item 122, 124, 324, 326, 328 with the provided encryption key and may transmit the reencrypted health data item to the customer.

In a still further example, the download validation operation 150, 156 may additionally or alternatively include checking the purchased health data item for corruption. In certain implementations, it is possible for the health data item 122, 124, 324, 326, 328 to degrade over time during storage. However, because absolute accuracy of the health data item 122, 124, 324, 326, 328 is important to properly perform medical research, it may be necessary to ensure that any such degraded health data items 122, 124, 324, 326, 328 are not provided to customers. Accordingly, the validator 148, 152 in connection with the download validation operation 150, 156 may analyze the accessed health data item 122, 124, 324, 326, 328 to determine whether it has been corrupted. For example, the validator 140, 152 may use a checksum stored with the health data item 122, 124, 324, 326, 328 to check whether the health data item has been corrupted during storage. In certain implementations, the checksum may be stored with phenotypic data 128 of the health data item 122, 124, 324, 326, 328.

After completing the download validation operation 150, 156, the validator 140, 152 may generate a download validation transaction 432 (block 706). The download validation transaction 432 may include a transaction ID 434 indicating a unique identifier for the specific download validation transaction 432 being generated. The download validation transaction 432 may also include a data item ID 436, similar to the data item IDs 406, 416, 426 discussed above, which may include an identifier or identify errors for the health data item or items 122, 124, 324, 326, 328 being validated by the download validation operation 150, 156 performed at block 704. The download validation transaction 432 may also include a user ID 438, which may correspond to a user ID of the entity implementing the validator 148, 152 or user ID corresponding to the validator 142, 152 itself, similar to the user ID 418 discussed above in connection with the upload validation transaction 412. Further, the user ID 438 may, in certain implementations, be anonymized prior to inclusion within the download validation transaction 432, as discussed above.

The download validation transaction 432 may also include a download validation result 440. The download validation result 440 may include an indication of the result of the download validation operation 150, 156. For example, in instances where the download validation 150, 156 determines that the accessed health data item is no longer stored within the health data storage 120, 322, the download validation result 440 may indicate that the health data item is missing. Additionally, in instances where the download validation operation 150, 156 determines that the health data item 122, 124, 324, 326, 328 has been corrupted during storage, the download validation result 440 may indicate that the health data item is corrupted. However, in instances where the data is successfully validated, the download validation result 440 may indicate that the health data item 122, 124, 324, 326, 328 was successfully validated. Further, where the download validation operation includes additional steps, such as transmitting the health data item to the customer, or providing re—of access to the customer, the download validation result 440 may indicate the subsequent steps taken and/or access granted to the customer.

Validator 148, 152 may then store the download validation transaction 432 on the blockchain 112, 302 (block 708). As discussed above in connection with blocks 508, 516, 612, the validator 148, 152 may transmit the download validation transaction 432 to the nodes 114, 116, 118 implementing the blockchain 112, 302. The nodes 114 and 16118 may group the download validation transaction 432 with other transactions 402, 412, 422, 432 to form a block 308. The nodes 114, 116, 118 may then work to determine the correct hash value 310 for the block 308 according to one or more consensus properties of the blockchain 112, 302. The first node 114, 116, 118 to identify the correct hash value 310 may then transmit the completed block 308 including the correct hash value 310 to the other nodes 114, 116, 118 for verification. After the block 308 is verified by the other nodes, 114, 116, 118, the block 300 and may then be appended to the end of the blockchain 112, 302.

Performing the method 700 may enable the system 100 and the validators 148, 152 to ensure the quality of the health data items 122, 124, 324, 326, 328 being downloaded or accessed from the health data storage 120, 322. This validation may help ensure that the system 100 is able to store and provide health data items 122, 124, 324, 326, 328 useful for medical research, which may in turn encourage customers to purchase access to the health data items 122, 124, 324, 326, 328 stored on the health data storage 120, 322, thereby further incentivizing users to upload health data items 122, 124, 324, 326, 328.

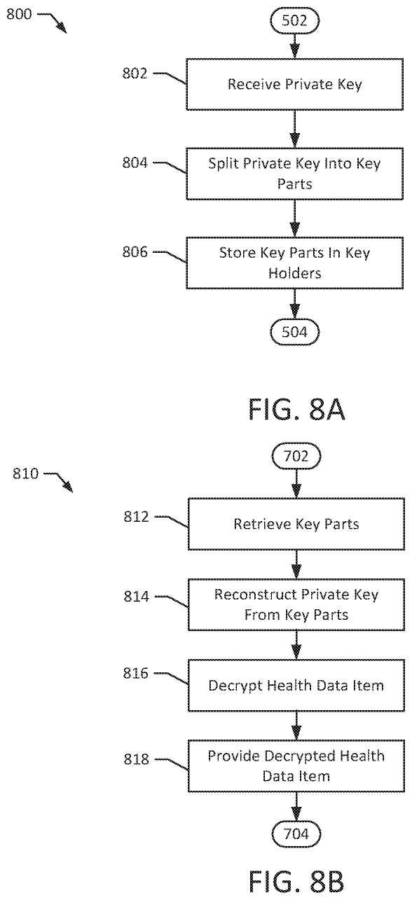

FIG. 8A depicts a flowchart of a method 800 for managing encryption keys according to an exemplary embodiment of the present disclosure. In certain implementations, the system 100 and the key management system 130, 200 may be configured to perform the steps of the method 800 in connection with a health data item upload procedure. For example, as depicted, the method 800 may be performed after receiving a health data item (block 502) and before storing a health data item on a health data storage (block 504). Alternatively, the method 800 may be performed simultaneously with storing the health data item 122, 124, 324, 326, 328 on the health data storage 120, 322 (block 504). The method 800 may be performed by the key management system 130 to store and manage encryption keys that may be used to encrypt health data items 122, 124, 324, 326, 328 prior to storage on the health data storage 120, 322.

The method 800 may be implemented on at least one computer system. For example, one or more steps of the method 800 may be implemented by the key management system 130, 200 and the user portal 110. Although the examples below are described with reference to the flowchart illustrated in FIG. 8A, many other methods of performing the acts associated with FIG. 8A may be used. For example, the order of some of the blocks may be changed, certain blocks may be combined with other blocks, one or more of the blocks may be repeated, and some of the blocks described may be optional.

The method 800 begins with the key management system 130, 200 receiving a private key (block 802). The private key may be a private key 208 from a key pair 204 associated with the user device 108 and used to encrypt a health data item 122, 124, 324, 326, 328 prior to storage on a health data storage 120, 322. For example, the private key 208 may be used to encrypt the health data item 122, 124, 324, 326, 328 received a block 502. The private key 208 may be received from a user portal 110, and may be transmitted via the network 102. In certain implementations, the private key 208 may itself be encrypted prior to transmission to the key management system 130, 200 via the network 102.

The key management system 130, 200 may then split the private key 208 into a plurality of key parts 216, 218, 220 (block 804). The private key 208 may be split into key parts 216, 218, 220 by a key splitter 214 of the key management system 130, 200. In certain implementations, the key management system 130, 200 may include a plurality of key holders 132, 138, 224, 228, 232. In such implementations, the key splitter 214 may split the private key 208 into the same number of key parts 216, 218, 220 as there are key holders 132, 138, 224, 228, 232 within the key management system 130, 200. In certain implementations, the system 100 may be configured to operate without key splitting. For example, the system 100 may be configured to store the private key 208 incomplete form (e.g., in a single key holder 132, 130, 224, 228, 232). Additionally, in other implementations, the number of key parts into which the private key 208 is split may be updated and adjusted to meet system requirements (e.g., security requirements, performance requirements).

The key management system 130, 200 may then store the key parts 216, 218, 220 in the key holders 132, 138, 224, 228, 232 (block 806). In implementations where the private key 208 is split into the same number of key parts 216, 218, 220 as there are key holders 132, 138, 224, 228, 232, the key management system 130, 200 may store one key part 216, 218, 220 on each key holder 132, 138, 224, 228, 232. As discussed above, the key parts may be encrypted using an encryption key associated with each key holder 132, 138, 224, 228, 232 prior to storage in the key holder 132, 138, 224, 228, 232. Accordingly, the key holders 132, 138, 224, 228, 232 may store the encrypted key parts 226, 230, 234 corresponding to the key parts 216, 218, 220. In other implementations, more than one key part 216, 218, 220 may be stored on each key holder 132, 138, 224, 228, 232. For example, certain key holders 132, 138, 224, 228, 232 may store two, three, or more key parts 216, 218, 220, and certain key holders 132, 138, 224, 228, 232 may store all of the key parts 216, 218, 220 for a given private key 208. Such implementations may be selected for, e.g. increased speed or reduced complexity of the system. After performing the steps of the method 800, the private key 208 may be securely stored for later use (e.g., in decrypting the health data item 122, 124, 324, 326, 328 for access by a customer). Processing may then continue through the health data item upload process, such as by storing the encrypted health data item on the health data storage 120, 322 at block 504.

FIG. 8B depicts a flowchart of a method 810 for managing encryption keys according to an exemplary embodiment of the present disclosure. In certain implementations, the system 100 and the key management system 130, 200 may be configured to perform the steps of the method 810 in connection with a health data access procedure. For example, as depicted, the method 810 may be performed after retrieving the health data item 122, 124, 324, 326, 328 (block 702) and before validating the health data item 122, 124, 324, 326, 328 with a validator 148, 152 (block 704). Alternatively, the method 810 may be performed simultaneously with retrieving the health data item 122, 124, 324, 326, 328 (block 702). The method 810 may be performed by the key management system 130, 200 and a validator 148, 152 to decrypt an encrypted health data item prior to validation, e.g., by a download validation operation 150, 156. In certain implementations, the method 810 may be performed in conjunction with the method 800 to implement one or more features of the key management system 130, 200. For example, the method 810 may be used to retrieve a private key 208 stored as multiple key parts 216, 218, 220 in key holders 132, 138, 224, 228, 232, as described above in connection with the method 800.

The method 810 may be implemented on at least one computer system. For example, one or more steps of the method 800 may be implemented by the key management system 130, 200, the validator 148, 152, and the customer portal 106. Although the examples below are described with reference to the flowchart illustrated in FIG. 8B, many other methods of performing the acts associated with FIG. 8B may be used. For example, the order of some of the blocks may be changed, certain blocks may be combined with other blocks, one or more of the blocks may be repeated, and some of the blocks described may be optional.

The method 810 may begin with the key management system 130, 200 retrieving the key parts from the key holders 132, 138, 224, 228, 232 that correspond to the health data item 122, 124, 324, 326, 328 being accessed (block 812). For example, in implementations where one key part 216, 218, 220 is stored separately in each key holder 132, 138, 224, 228, 232, the key management system 130, 200 may retrieve one key part 216, 218, 220 from each key holder 132, 138, 224, 228, 232 corresponding to the accessed health data item 122, 124, 324, 326, 328. For example, the key management system 130, 200 may provide the data item ID 436 from a download validation transaction 432 to the key holders 132, 138, 224, 228, 232, which may then provide the key parts 216, 218, 220 associated with that data item ID 436. In certain implementations, the key parts 216, 218, 220 may be stored as encrypted key parts 226, 230, 234, as discussed above. In such implementations, the key holders 224, 220, 232 may decrypt the encrypted key parts 226, 230, 234 prior to providing the key parts 216, 218, 220 to the key management system 130, 200.

Once the key parts 216, 218, 220 are retrieved, the key management system 130, 200 then reconstruct the private key 208 from the key parts 216, 218, 220 (block 814). For example, the key management system 130, 200 may reorder the received key parts 216, 218, 220 into the correct order to re-create the private key 208. For example, the key holders 132, 138, 224, 228, 232 may store ordering indicators with the encrypted key parts 226, 230, 234, which may be used to properly order the key parts 216, 218, 220 after decryption. In another example, the correct ordering may be shared between the key management system 130, 200 and the key holders 224, 228, 232 using the Shamir Secret Sharing protocol. The key management system 130 may then decrypt the encrypted health data item with the reconstruct the private key 208 (block 816). For example, the key management system 130 may decrypt the health data item using a decryption operation according to the encryption scheme used to encrypt the health data item 122, 124, 324, 326, 328.

The key management system 130, 200 may then provide the decrypted health data item 122, 124, 324, 326, 328 for use in future processing (block 818). For example, where the method 810 is performed in connection with a download validation operation 150, 156, the key management system 130, 200 may provide the decrypted health data item 122, 124, 324, 326, 328 to the validator 148, 152 performing the download validation operation 150, 156 in connection with block 704 discussed above. For example, the key management system 130 may transmit the decrypted health data item to the validator 140, 152 via the network 102.

Although the above-discussed operations were described as being performed by the key management system 130, in certain implementations, one or more operations of the method 810 may be performed by the validator 148, 152 performing the download validation operation 150, 156. For example, the validator 140, 152 may perform the above-describe steps in connection with retrieving the health data item at block 702. In such implementations, block 818 may not be necessary, as the validator 148, 150 to perform the download validation operation 150, 156 may already have the decrypted health data item 122, 124, 324, 326, 328 for future processing at block 704.

In performing the method 810, the key management system may be able to securely store the key parts will also retrieving them as needed for validation and customer access purposes. Accordingly, the method 810 in connection with the method 800 may enable the system 100 to store health data items 122, 124, 324, 326, 328 in a method compliant with HIPAA and GDPR and other medical data privacy regulations.

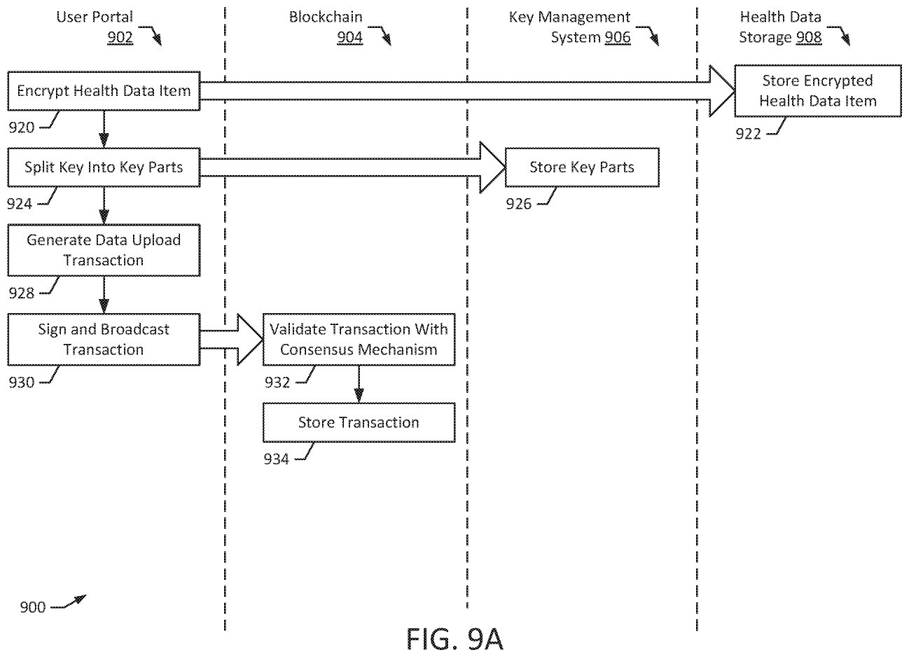

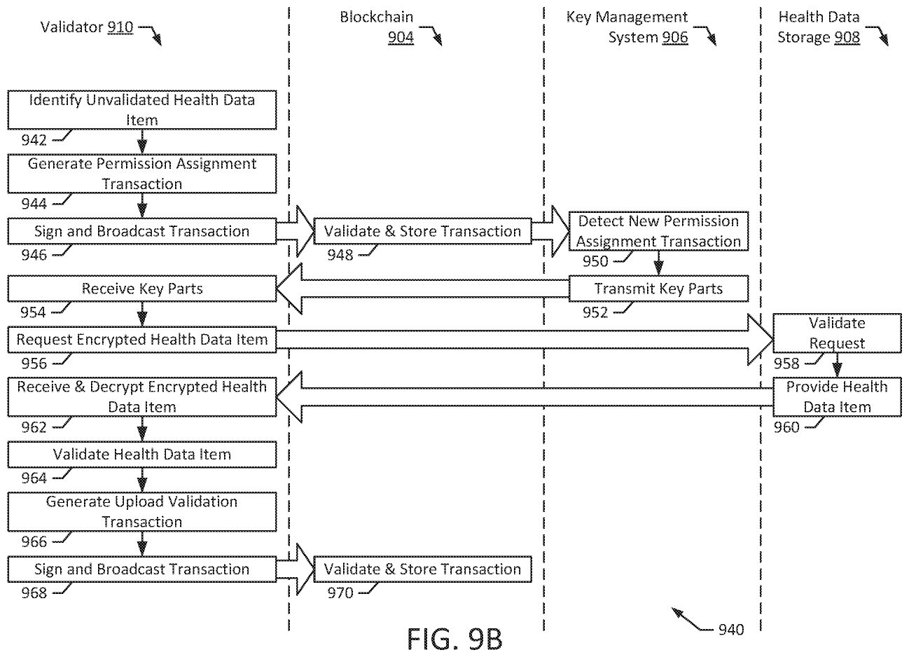

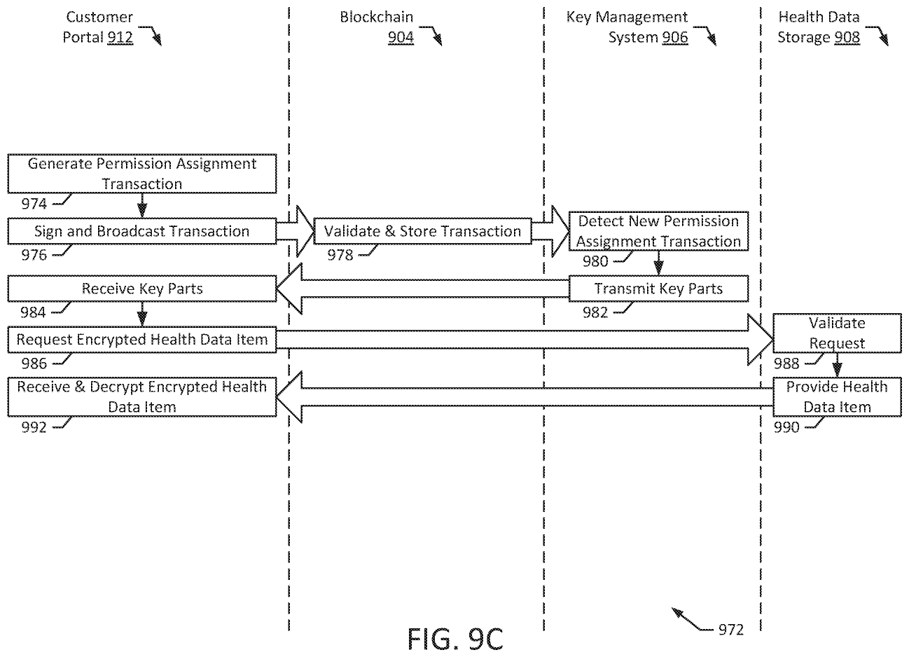

FIGS. 9A-9C depict a plurality of methods 900, 940, 972 according to exemplary embodiments of the present disclosure. The methods 900, 940, 972 may be implemented on a computer system, such as the system 100. For example, the methods 900, 940, 972 may be implemented by the customer portal 106, the user portal 110, the validators 144, 148, 152, 236, 238, 240, the key management system 130, and the health data storage 120, 322. The methods 900, 940, 972 may also be implemented by a set of instructions stored on a computer readable medium that, when executed by a processor, cause the computer system to perform the method. For example, all or part of the method 900, 940, 972 may be implemented by one or more CPU and memories implementing the customer portal 106, the user portal 110, the validators 144, 148, 152, 236, 238, 240, the key management system 130, and the health data storage 120, 322. Although the examples below are described with reference to the flowchart illustrated in FIGS. 9A-9C, many other methods of performing the acts associated with FIGS. 9A-9C may be used. For example, the order of some of the blocks may be changed, certain blocks may be combined with other blocks, one or more of the blocks may be repeated, and some of the blocks described may be optional.

FIG. 9A depicts a method 900 that may be executed to upload a health data item 122, 124, 324, 326, 328 for storage on a health data storage 120, 322. FIG. 9A includes a user portal 902, which may be an example implementation of the user portal 110, a blockchain 904, which may be an example implementation of the blockchain 112, 302, a key management system 906, which may be an example implementation of the key management system 130, and a health data storage 908, which may be an example implementation of the health data storage 120, 322.

The method 900 begins with the user portal 902 encrypting a health data item 122, 124, 324, 326, 328 (block 920). The user portal may use a symmetric cipher (e.g., Advanced Encryption Standard (AES-256) algorithm in the cipher blocker chaining (CBC) mode, or the XSalsa20 algorithm with a Poly1305 message authentication code) or an asymmetric cipher (e.g., Rivest-Shamir-Adleman (RSA) protocol). The user portal 902 may use a key pair 204, 210, 212 to encrypt the health data item 122, 124, 324, 326, 328.

The user portal 902 may then transmit the encrypted health data item 122, 124, 324, 326, 328 to the health data storage 908, which may then store the encrypted health data item 122, 124, 324, 326, 328 (block 922). As discussed above, the health data storage 908 stores encrypted health data items 122, 124, 324, 326, 328 to protect the privacy of the user uploading the health data item 122, 124, 324, 326, 328.

The user portal 902 may then split the key pair 204, 210, 212 used to encrypt the health data item 122, 124, 324, 326, 328 into a plurality of key parts 134, 136, 140, 142, 216, 218, 220 (block 924). The key splitting may be performed by a key splitter 214. The user portal 902 may then transmit the key parts 134, 136, 140, 142, 216, 218, 220 to the key management system 906, which may then store the key parts (block 926). The key management system 906 may include one or more key holders 132, 138, 224, 228, 232 configured to store key parts 134, 136, 140, 142, 216, 218, 220. In certain implementations, the key holders 132, 138, 224, 228, 232 are configured to store encrypted key parts 226, 230, 234. For example, prior to transmitting the key parts 134, 136, 140, 142, 216, 218, 220, the user portal 210 may encrypt the key parts 134, 136, 140, 142, 216, 218, 220 using a public key associated with key holders 132, 138, 224, 228, 232 of the key management system 906 to form encrypted key parts 226, 230, 234. Further, each key holder 132, 138, 224, 228, 232 may be configured to store a single encrypted key part 226, 230, 234. In such implementations, the user portal 902 and the key splitter 214 may split and encrypt the key pair 204, 210, 212 into the same number of encrypted key parts 226, 230, 234 as there are key holders 132, 138, 224, 228, 232 of the key management system 906.

The user portal 902 may then generate a data upload transaction 402 (block 928). The data upload transaction 402 may include one or more of a transaction ID 404 identifying the data upload transaction 402, a data item ID 406 (e.g., a hash of the health data item 122, 124, 324, 326, 328) that identifies the uploaded health data item 122, 124, 324, 326, 328, a user ID 408 identifying the user uploading the health data item 122, 124, 324, 326, 328 via the user portal 902, and a data type 410 identifying a data type of the health data item 122, 124, 324, 326, 328.

The user portal 902 may then sign and broadcast the data upload transaction 402 to the blockchain 904 (block 930). After receiving the signed data, the blockchain 904 may then validate the transaction with the consensus mechanism (block 932). For example, as described above, one or more nodes 114, 116, 118 implementing the blockchain 904 may perform the consensus algorithm on the data upload transaction 402 to verify that the data upload transaction 402 is authentic. If the blockchain 904 successfully validates the data upload transaction 402, the data upload transaction 402 may then be stored on the blockchain 904 (block 934).

FIG. 9B depicts a method 940 which may be performed to validate an uploaded health data item 122, 124, 324, 326, 328 using a validator 144, 148, 152, 236, 238, 240. In addition to the blockchain 904, key management system 906, and health data storage 908, FIG. 9B includes a validator 910, which may be implemented by the validator 144, 148, 152, 236, 238, 240.

The method 940 begins with the validator 910 identifying an unvalidated health data item 144, 148, 152, 236, 238, 240 (block 942). The validator 910 may identify the unvalidated health data item by analyzing transactions 312, 314, 316, 402, 412, 422, 432 stored on the blockchain 904. For example, the validator may identify an unvalidated health data item 144, 148, 152, 236, 238, 240 by identifying a data upload transaction 402 on the blockchain 904 with no corresponding permission assignment transaction 422 or upload validation transaction 412.

The validator 910 may then generate a permission assignment transaction 422 for the unvalidated health data item 144, 148, 152, 236, 238, 240 (block 944). The permission assignment transaction 422 may assign permission to the validator 910 to access the health data item 144, 148, 152, 236, 238, 240 in order to validate the health data item 144, 148, 152, 236, 238, 240 after upload by the user portal 902. The permission assignment transaction 424, data item ID 426, customer ID 428, and permission level 430 as discussed above in FIG. 4.

The validator 910 may then sign and broadcast the permission assignment transaction 422 (block 946) to a blockchain 904 for validation with a consensus algorithm of the blockchain 904 and storage on the blockchain 904 (block 948). In certain implementations, the validator 910 may purchase access to the health data item 122, 124, 324, 326, 328 for validation purposes in, e.g., cryptocurrency or fiat currency. In addition to validating the authenticity of the permission assignment transaction 422, the blockchain 904 may also validate that the validator 910 has provided the currency for purchasing access to the health data item 122, 124, 324, 326, 328 (e.g., has provided sufficient currency to a smart contract application running on the blockchain 904).

The key management system 906 may then detect the new permission transaction 422 stored on the blockchain 904 (block 950). In response to detecting the new permission transaction 422, the key management system 906 may then transmit the encrypted key parts 226, 230, 234 to the data validator 910 (blocks 952, 954). The data validator 910 may decrypt the encrypted key parts 226, 230, 234 to obtain the key parts 134, 136, 140, 142, 216, 218, 220 and reconstitute the key pair 204, 210, 212.

The validator 910 may also request the encrypted health data item 122, 124, 324, 326, 328 from the health data storage 908 (block 956). For example, the validator 910 may request the encrypted health data item 122, 124, 324, 326, 328 via an encrypted communication channel between the validator 910 and health data storage 908. The health data storage 908 then validates the request (block 958) and provides the encrypted health data item 122, 124, 324, 326, 328 to the data validator 910 if the request is successfully validated (block 960). The health data storage 908 may validate the request to confirm that the validator 910 has permission to access the requested health data item 122, 124, 324, 326, 328. For example, the health data storage 908 may analyze the blockchain 904 for a permission assignment transaction 422 granting the validator 910 permission to access the requested health data item 122, 124, 324, 326, 328. In certain implementations, if the validator 910 takes too much time to validate the health data item 122, 124, 324, 326, 328, it may be determined that the health data item 122, 124, 324, 326, 328 is valid or invalid based on a default setting of, e.g., the validator 910 or a smart contract controlling the interactions of the validator 910 with the system 100.

After receiving the encrypted health data item 122, 124, 324, 326, 328, the validator 910 may then decrypt the encrypted health data item 122, 124, 324, 326, 328 (block 962). The validator 910 may decrypt the encrypted health data item 122, 124, 324, 326, 328 using the key pair 204, 210, 212 reconstituted at block 954. The validator 910 may then validate the health data item 134, 136, 140, 142, 216, 218, 220 (block 964). As discussed above, the validator 910 may validate the health data item using, e.g., an upload validation operation 146, 154. If the validator 910 is unable to validate the health data item 122, 124, 324, 326, 328, the validator 910 may be returned any currency exchanged for access to the health data item 122, 124, 324, 326, 328 and the health data item 122, 124, 324, 326, 328 may be prevented from being available for sale to other users (e.g., customers using a customer portal 106.

After validating the health data item, the validator 910 may generate an upload validation transaction 412 indicating that the health data item 122, 124, 324, 326, 328 has been validated (block 964). As described above in connection with FIG. 4, the upload validation transaction 412 may include a transaction ID 414, a data item ID 416, a user ID 418, and an upload validation result 420. In certain implementations, the upload validation transaction 412 may also include a hash value of the health data item 122, 124, 324, 326, 328 and the validation result generated by the validator 910 in performing the upload validation operation 146, 154. The validator 910 may then sign and broadcast the upload validation transaction 412 to the blockchain 904 (block 968), which may then validate and store the transaction 412 (block 970).

After validating the data, the validator 910 may be entitled to a certain portion of the currency received to access the validated health data item 122, 124, 324, 326, 328 moving forward. For example, the validator 910 may receive a set percentage (e.g., 5%, 10%, 30%) for validating the health data item 122, 124, 324, 326, 328. In certain implementations, more than one validator 910 may validate a single health data item 122, 124, 324, 326, 328. In such implementation, the validators 910 may split the received portion of the currency received to access the validated health data item 122, 124, 324, 326, 328.

FIG. 9C depicts a method 972 that may be performed to provide access to a health data item 122, 124, 324, 326, 328 to a customer portal 912. In addition to the blockchain 904, key management system 906, and health data storage 908, FIG. 9C also includes a customer portal 912, which may be implemented by the customer portal 106.

The method 972 may begin with the customer portal 912 generating a permission assignment transaction 422 (block 974). The customer portal 912 may identify one or more health data items 122, 124, 324, 326, 328 that may be useful to the customer for, e.g., performing medical research or providing medical advice or treatment. The customer portal 912 may identify the health data items 122, 124, 324, 326, 328 using, e.g., a smart contract that runs on the blockchain 904. The permission assignment transaction 422 may include a transaction ID 424, a data item ID 426, a customer ID 428, and a permission level 430. The customer portal 912 may then sign and broadcast the permission assignment transaction 422 to the blockchain 904 (block 976), which may then validate and store the permission assignment transaction 422 (block 978). As described above in connection with block 948, the customer portal 912 may purchase access the health data item 122, 124, 324, 326, 328 using cryptocurrency or fiat currency and the blockchain 904 may also validate that the currency has been transferred (e.g., to the user or to a smart contract running on the blockchain 904).

The key management system 906 may then detect a new permission assignment transaction 422 on the blockchain 904 (block 980) and may transmit the encrypted key parts 226, 230, 234 to the customer portal 912 (block 982). The customer portal 912 may receive and decrypt the encrypted key parts 226, 230, 234 to obtain the key parts 134, 136, 140, 142, 216, 218, 220 and reconstitute the key pair 204, 210, 212 (block 984).

The customer portal 912 may also request the encrypted health data item 122, 124, 324, 326, 328 from the health data storage 908 (block 986) and the health data storage 908 may then validate the request (block 988) and provide the health data item 122, 124, 324, 326, 328 if the request is successfully validated (block 990). The customer portal 912 may then receive and decrypt the encrypted health data item 122, 124, 324, 326, 328 using the reconstituted key pair 204, 210, 212 (block 992).

In certain implementations, rather than receiving the encrypted key parts 226, 230, 234 and encrypted health data item 122, 124, 324, 326, 328 directly, the customer portal 912 may instead rely on other components of the system. For example, as discussed above, a validator 908 may instead receive the encrypted key parts 226, 230, 234 and encrypted health data item 122, 124, 324, 326, 328. The validator 908 may decrypt the health data item 122, 124, 324, 326, 328 by reconstituting the key pair 204, 210, 212 and may validate the health data item 122, 124, 324, 326, 328 using a download validation operation 150, 156. The validator may generate a download validation transaction 432 and may then provide the health data item 122, 124, 324, 326, 328 to the customer portal 912 (e.g., by encrypting the 122, 124, 324, 326, 328 with a public key of the customer portal 912). In another example, the key management system 906 may decrypt the encrypted health data 122, 124, 324, 326, 328 item using the encrypted key parts 226, 230, 234 stored on the key holders 132, 138, 224, 228, 232 and may similarly provide the health data item to the customer portal 912.

One or more of the blocks 980, 982, 984, 986, 988, 990, 992 may be implemented as discussed above in connection with blocks 950, 952, 954, 956, 958, 960, and 962 of the method 940.

### Additional System Descriptions

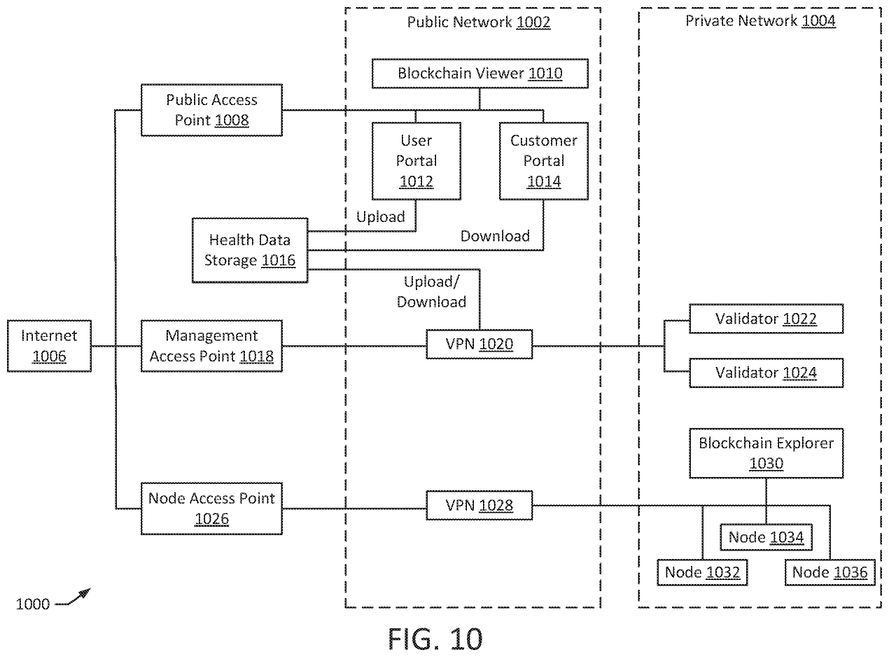

FIG. 10 depicts a blockchain of a system 1000 according to an exemplary embodiment of the present disclosure. The system 1000 may depict one or more network configurations of the system 100, such as configurations of the network 102. One or more components of the system 1000 may be implemented by a computer system. For example, one or more components of the system 1000 may be implemented by a memory storing instructions which, when executed by a processor, cause the processor to perform one or more of the operational features of the one or more components.

The system 1000 includes a public network 1002 and a private network 1004. The public network 1002 may be a network to which various public-facing applications and access points connect. In certain implementations, the public network 1002 may be directly accessible one or more access points via the Internet. For example, the public network 1002 may be accessible via the public access point 1008, management access point 1018, and node access point 1026, which are connected to the Internet 1006. The private network 1004 may not be directly accessible via public connections, but may instead require connection via one or more virtual private networks (VPNs) 1020, 1028. The VPNs 1020, 1028 may, in certain implementations, connect to the private network 1004 indirectly via one or more network address translation (NAT) services, such as a NAT balancer (not depicted).

The public access point 1008 may be used by public users of the system 1000, such as customers and users, to upload and access health data items 122, 124, 324, 326, 328. For example, the public access point 1008 may be used by a user portal 1012 (which may be an implementation of the user portal 110) or a customer portal 1014 (which may be an implementation of the customer portal 106) to upload health data items 122, 124, 324, 326, 328 or to purchase access to health data items 122, 124, 324, 326, 328. As depicted, the user portal 1012 and customer portal 1014 are connected to the public network 1002 via the public access point to perform these functions. Additionally, the user portal 1012 and customer portal 1014 are connected to a health data storage 1016 (which may be an implementation of the health data storage 120, 322, 908), with the user portal 1012 connected with an upload-only connection and the customer portal 1014 connected with a download-only connection. These connections may be utilized to, e.g., upload health data items 122, 124, 324, 326, 328 from the user portal 1012 and download authorized health data items 122, 124, 324, 326, 328 for which access permissions have been granted. The public access point 1008 is also connected to a blockchain viewer 1010 on the public network 1002, which may be used to access and analyze the transactions 312, 314, 316, 402, 412, 422, 432 stored on the blockchain 112, 302, 904. For example, one or both of the user portal 1012 and the customer portal 1014 may connect to the blockchain viewer 1010 to enable users and customers to analyze transactions 312, 314, 316, 402, 412, 422, 432.

The management access point 1018 may be accessible only to administrators of the system, such as administrators of the validators 1022, 1024. The validators 1022, 1024 may themselves be implementations of the validators 144, 148, 152, 236, 238, 240, 910. To enhance privacy and security, the validators 1022, 1024 may be connected to the private network 1004 and may only connect to the public network 1002 and the management access point 1018 using the VPN 1020, as also described previously. The VPN 1020 may also connect to the health data storage 1016 with an upload and download link to enable the validators 1022, 1024 to download health data items 122, 124, 324, 326, 328 to be validated and to upload health data items 122, 124, 324, 326, 328 that have been validated.

The node access point 1026 may be accessed by one or more nodes of the blockchain 112, 302, 904. The node access point 1026 connects to a VPN 1028 in the public network 102. The VPN 1028 then connects to nodes 1032, 1034, 1036, which may be additional nodes 1032, 1034, 1036 implementing a blockchain 112, 302, 904. The VPN 1028 also connects to a blockchain 1030, which may be used to explore one or more of the nodes 1032, 1034, 1036 implementing the blockchain 112, 302, 904, validators 1022, 1024 and other users accessing the blockchain 112, 302, 904, and transactions 312, 314, 316, 402, 412, 422, 432 stored on the blockchain 112, 302, 904. In certain implementations, the blockchain explorer 1030 may enable one or more features of the blockchain viewer 1010.

### Data Valuation

To encourage provision of health data items 122, 124, 324, 326, 328 by users, customers may purchase access to health data items 122, 124, 324, 326, 328 in exchange for currency and/or cryptocurrency. As described above, the purchase price may be shared with the validator or validators 144, 148, 152, 236, 238, 240, 910 that helped validate the health data item 122, 124, 324, 326, 328. To facilitate these transactions, a price may be determined for one or more health data items 122, 124, 324, 326, 328 stored on the health data storage 120, 322, 908.

The purchase price paid may vary depending on multiple factors, including the type of health data item 122, 124, 324, 326, 328 (e.g., basic blood tests, basic urine tests, MRI scans, electroencephalograms, electrocardiograms, genome sequences, transcriptomes, microbiome evaluations), the quality of the health data item 122, 124, 324, 326, 328 as determined by a validator 144, 148, 152, 236, 238, 240, 910, a scarcity or commonality of health data items 122, 124, 324, 326, 328 of a similar type, a biological significance of the health data item 122, 124, 324, 326, 328 for diagnosing or studying certain disease conditions, and an amount of access given to the health data item 122, 124, 324, 326, 328. Different types of health data items 122, 124, 324, 326, 328 may have their own predictive value, representative sensitivity, prediction rate, and weight. Additionally, doctors and other health professionals may be better able to diagnose or monitor a patient' health with health data items 122, 124, 324, 326, 328 of different types. For example, traditional diagnostic pipelines are based on analysis combination of medical tests, especially when healthcare specialists try to diagnose serious and complex pathologies such as oncological, autoimmune, or neurodegenerative diseases. Combining multiple data types, especially low-level diagnostics data, provides a multi-level overview and better understanding of complex multifactorial conditions, and also leads to faster diagnostics of patient conditions.

Medical researchers are also constantly searching for suitable groups of biomarkers based on the multi-level data for medical conditions. These biomarkers may take multiple forms and researchers commonly use medical tests and other health data items 122, 124, 324, 326, 328 for broader diagnostic applications than initially intended. Based on these changes, the value and price of a health data item 122, 124, 324, 326, 328 of a specific type for medical research may change overtime.

Also, despite a large number of various diagnostic tests, not all types of medical data or health data items 122, 124, 324, 326, 328 may be desirable for analyzing or predicting a user's changing health over time. For example, genome analysis may provide important information on heredity, but due to its relative stability may have a low value for prediction of dynamic changes in a patient compared to epigenome or transcriptome.

Further, combining health data items 122, 124, 324, 326, 328 of different types may provide robust datasets helpful for use within artificial intelligence (AI) models. For example, an AI model may be used to identify biomarkers for diseases, as discussed above, or may be used to diagnose diseases within a patient based on health data items 122, 124, 324, 326, 328 of different types.

To facilitate the acquisition and combination of such health data items 122, 124, 324, 326, 328, including health data items 122, 124, 324, 326, 328 of different types, a price may be generated for health data items 122, 124, 324, 326, 328 individually or in combination such that a customer may purchase access to the health data items 122, 124, 324, 326, 328 (e.g., through a customer portal 106, 912).

### Data Value Model

Generally, health data items 122, 124, 324, 326, 328 can be divided into the following categories: dynamic—reflecting the state of a patient or organism at the time of sampling (e.g., blood tests, transcriptomes, epigenomes, proteomes, microbiome evaluations), and static—almost unchanged during the life of the patient or organism (e.g., genomes, fingerprints). Within a dynamic group, it is possible to differentiate rapidly changing data and gradually changing data.

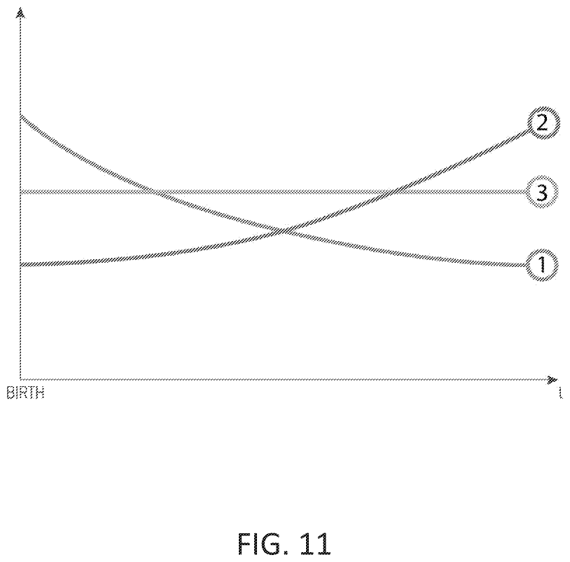

For example, FIG. 11 depicts the relative value of certain health data items 122, 124, 324, 326, 328 from different periods of a patient's life. For congenital diseases, health data items 122, 124, 324, 326, 328 obtained in the first years of a patient's life may be important as determining the further development of the disease (e.g., line 1 of FIG. 11). By contrast, for age-associated diseases, it may be more important to analyze the health data items 122, 124, 324, 326, 328 obtained before a diagnosis was made (e.g., line 2 of FIG. 11), and health data items 122, 124, 324, 326, 328 of a type that tends to remain constant throughout the life of the patient (e.g., line 3 of FIG. 11).

In pricing the health data items 122, 124, 324, 326, 328, each record of the health data items 122, 124, 324, 326, 328 could be viewed as a triplet (type, time, quality), where type may be a categorical variable for a record type of the health data item health data items 122, 124, 324, 326, 328, time is a sampling time of patient's biomedical record (e.g., when a blood test was performed) minus the patient's time of birth, and quality is a nonnegative number reflecting record quality (e.g., a vector generated by a validator 144, 148, 152, 236, 238, 240, 910). The type may also include a half-life period of analysis for the health data item 122, 124, 324, 326, 328, which may characterize the half-duration of the relevance of the health data item 122, 124, 324, 326, 328. For example, a cholesterol check may be valid for only five years or less if a patient is at higher risk for heart disease, while a genome profile may be valid for the whole life of the patient. Thus, genome analysis may have a longer half-life period than basic cholesterol blood test.

For the foregoing discussions, the dataset may be implemented as a setDataset={(userₘ, Rn)} n=1 of N (userₘ, Rn) pairs, where user is a user profile. A user profile (Patient's profile) may be an attribute that includes health information such as ethnicity, date of birth, sex, diagnoses, blood type, medical prescriptions, vaccinations, chronic diseases, interventions, smoking, and alcohol status, family relations, weight, height, geolocation. User profile may also refer be considered a hybrid attribute if it includes both static (e.g., date of birth, ethnicity, sex, blood type) and dynamic parameters (e.g., diagnoses, smoking and alcohol status, weight, geolocation).

The dataset Cost may a function of a Dataset and may consist of two terms: (i) a combination of health data items 122, 124, 324, 326, 328 for each single user and (ii) combinations for a set of same type health data items 122, 124, 324, 326, 328 for a group of users.

### Cost for Single User

One potential formula for determining a value of the health data items 122, 124, 324, 326, 328 for a user is:

$$Cost ( user ) = \text{?} \text{?} {f}_{k} ( {R}_{{i}_{s}} , … , {R}_{{i}_{k}} \vert user ) , \\ \text{?} \text{indicates text missing or illegible when filed}$$

where:

k is a number of records in a combination. In this case, all records in the combination may be for the user and may be different, and

fₖ is a cost function for a combination for k records, and where for k=1: R=(type, time, quality):

f₁(R|user)=Ψ(type|user),×quality×Ψ(type|user)

where:

Ψ (type|user) is a base value of given record type and user combination. In the model, the base value is set as a mapping of categorical parameter type to the positive numbers (0, ∞)

Ψ (type|user) is a time value of record. It is a function (0, ∞)→(0, ∞) such that

k>1:R₁=(type₁,time₁,quality₁), . . . R_{K}=(type_{K},time_{K},quality_{K}).

The cost function may be similar for a combination of several records or health data items 122, 124, 324, 326, 328 to the k=1 case above. In such a case, it may be necessary to define the base value, quality, and time value of an interaction component for the cost of several records.

fₖ(R₁, . . . ,Rₖ|user)=Ψₖ(type₁, . . . ,typeₖ|user)×vₖ(quality₁, . . . qualityₖ),×Ψₖ(time₁, . . . ,timeₖ,type₁, . . . ,typeₖ|user)

where

Ψₖ (type₁ . . . ,typeₖ|user) is a base value of addition due to interactions. It is a mapping of categorical parameters type₁, . . . , typeₖ to the positive numbers (0, ∞).

vₖ(quality₁, . . . qualityₖ) is a quality of combination of records for one user and may be a function [0, ∞)^{k}→[0, ∞) such that vₖ is monotonic nondecreasing function of each input and _{quality}lim_{m→0} vₖ(quality₁, . . . qualityₖ)=0 for all m=1, . . . k such that adding a record R with zero quality does not change the cost of the Dataset. For example, vₖ may be defined such that vₖ=(Σₘ₌₁^{k} 1/qualityₘ)⁻¹.

Ψₖ (time₁, . . . ,timeₖ, type₁, . . . , typeₖ|user) is a time value for a set of records. For a fixed set of time₁, . . . ,timeₖ Ψₖ (time₁, . . . ,timeₖ, type₁, . . . , typeₖ|user) may be a function [0, ∞)ₖ→[0, ∞).

For example, for each typeₘ, two time parameters Tₘ, type_{m,o} and a nonnegative function wₘ(t), t≥0 be chosen and Ψₖ (time₁, . . . , timeₖ, type₁, . . . , typeₖ|user) may be defined such that:

$${ψ}_{k} ( {time}_{1} , … , {time}_{k} , {type}_{1} , … , {type}_{k} \vert user ) = {max}_{t} {min}_{m} = 1 , {… }_{k} {w}_{m} ( \frac{t - {time}_{m , o}}{{T}_{m}} )$$

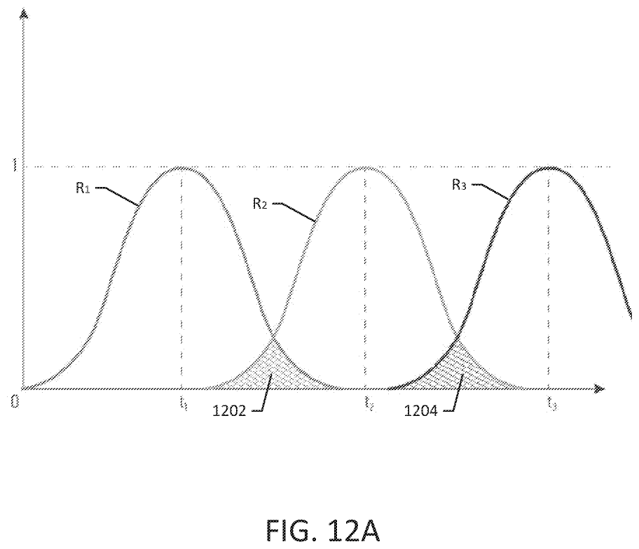

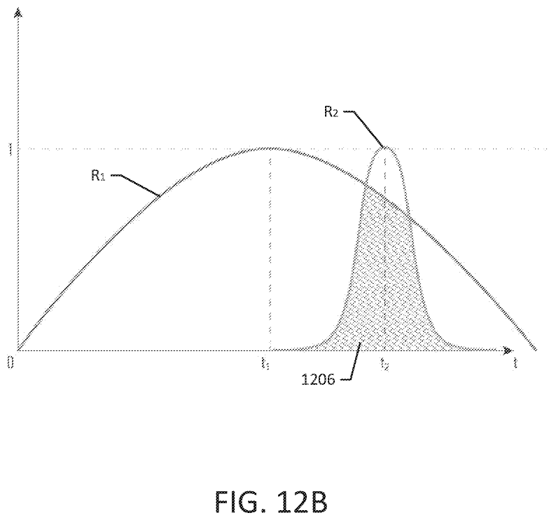

FIGS. 12A and 12B illustrate examples of a cost calculation for of a combination of health data items 122, 124, 324, 326, 328 depending on how old the health data items 122, 124, 324, 326, 328 are. More specifically, FIG. 12A depicts the cost of a combination of health data item 122, 124, 324, 326, 328 records R₁, R₂, R₃ of the same type obtained in different periods of time (e.g., blood tests made in different period of time) from a single patient, e.g., where type₁=type₂=type₃, quality₁=quality₂=quality₃, and time₁≠time₂≠time₃. FIG. 12B depicts the cost of a combination of health data item 122, 124, 324, 326, 328 records of different data types R₁, R₂ obtained at different periods of time (e.g., blood test and transcriptome analysis) from the single patient, where type₁≠type₂, quality₁=quality₂, time₁≠time₂.

In these Figures, the larger intersection 1202, 1204, 1206 of time value curves, the greater the combined records cost. As depicted, health data items 122, 124, 324, 326, 328 obtained in the same or a short period of time may have greater representation and predictive value. This concept may be understood and discussed herein as the time value of data—an indicator that demonstrates the representative and predictive rate of the group value of data, based on the difference in the records' half-life time. The time value of health data item 122, 124, 324, 326, 328 may be relevant both for combinations of health data item 122, 124, 324, 326, 328 of one type and for combinations of health data item 122, 124, 324, 326, 328 of different types.

### Cost for Records from a Group of Users

The cost of a combination of health data items 122, 124, 324, 326, 328 from multiple users may only increase for multiple records of the same type from distinct users. For example, fixing a type of record and letting userᵢ₁, . . . , userᵢₖ have records with type type in the Dataset and qualityᵢ₁, . . . qualityᵢₖ be the best corresponding qualities of user records with type in the Dataset. The cost for health data items 122, 124, 324, 326, 328 of the fixed type may be calculated according to:

$$Cost ( type , {quality}_{i 1 , … , } {quality}_{ik} , {user}_{i 1 , … } {user}_{ik} ) = \text{?} ( K , type \vert {user}_{i 1} , … , {user}_{ik} ) \times \frac{1}{K} {\sum }_{s = 1}^{K} {quality}_{ik} . \\ \text{?} \text{indicates text missing or illegible when filed}$$

where, for a fixed type function, γ(k, type|userᵢ₁, . . . , userᵢₖ) may have a fixed superlinear growth as k increases. For example, γ(k, type|useri1, . . . , userik) may be implemented as γ(k, type|useri1, . . . , userik)=C×K×lnk or γ(k, type|userᵢ₁, . . . , userᵢₖ)=C×K^{3/2}.

Each Dataset of health data item 122, 124, 324, 326, 328 may have its own critical representative level, which may depend on the type of health data items 122, 124, 324, 326, 328, the quality of health data items 122, 124, 324, 326, 328, and a patient profile used to select the health data item 122, 124, 324, 326, 328 for inclusion within the Dataset.

### Cost of Buying a Dataset

If a customer wants to buy a Dataset and already has bought some a portion of the Dataset (denoted Dataset¹), then the cost of the Dataset may be calculated according to:

Cost(Dataset)=Cost(Dataset∪Dataset¹)−Cost(Dataset¹)

The payments for user health data items 122, 124, 324, 326, 328 may then be fairly distributed among users according to their contribution to the Dataset cost and previous payments from the current customer.

### Family and Relationship Value of Data

Many medical studies require health data items 122, 124, 324, 326, 328 from closely related subjects (e.g., subjects from the same family or region), which may complicated the required data analysis. These challenges may be mitigated or overcome by powerful and efficient data analysis acquisition and design. For example, these issues may be alleviated by analyzing health data items 122, 124, 324, 326, 328 from genetically close patients, twins, siblings, or parents and offspring or colleagues and friends, where observed effects are influenced by a smaller number of potential patient features or biomarkers.

To identify such data, a coefficient of relationship (r) between two individuals, also known as a coefficient of inbreeding, may be calculated, where the coefficient of relationship between subjects B and C is defined as:

r_{BC}=Σp_{AB}p_{AC},

where p is for path coefficients connecting B and C with common ancestor A and

where p_{AB} is defined as:

$${p}_{AB} = {2}^{- n} \times \sqrt{( \frac{( 1 + {f}_{A} )}{( 1 + {f}_{B} )} )} ,$$

where f_{A} and f_{B} are inbreeding coefficient for ancestor A and offspring B, respectively.

Human populations are typically genetically heterogeneous and usually randomly-bred. Thus, the f_{A} may be set as f_{A}=0 and the formula for the coefficient of relationship could be simplified to:

r_{BC}=Σp2^{-L(p)}, where L(p) is the length of the path p.

In this implementation, r of a parent-offspring is 2⁻¹=0.5, and r of a grandparent-grandchild is 2⁻²=0.25.

The cost function of data could also be modified as:

fₖ(R₁, . . . ,Rₖ|user)=Ψₖ(type₁, . . . ,typeₖ|user)×vₖ(quality₁, . . . qualityₖ),×Ψₖ(time₁, . . . ,timeₖ,type₁, . . . ,typeₖ|user)+λ[Ψₖ(type₁, . . . ,typeₖ|user)×vₖ(quality₁, . . . qualityₖ),×Ψₖ(time₁, . . . ,timeₖ,type₁, . . . ,typeₖ|user)],

where λ is a regularization coefficient equal to a coefficient of relationship of users the system and could be set as:

λΣ^{m}ᵢ₌₁rᵢ,

where m is a number of users in the system and r is a coefficient of relationship between them.

For distant relatives, r→0 and almost will not contribute to the cost function of data, however, for a very close relative such as twins, r is equal to 1 and, at the beginning, will double the cost of data. The cost of data will grow with increases to the number of close relatives for which access to health data items 122, 124, 324, 326, 328 is sought.

### Patient Age Prediction

Chronological age is a feature possessed by every living organism and is one of the most important factors affecting the morbidity and mortality in humans. The multitude of biomarkers linked to disease are strongly correlated with age. For instance, triglycerides, glycated hemoglobin (HbA1c), waist circumference, IL-6 increase with age, but other parameters like albumin, IGF and creatinine clearance decrease with age. Combining various biomarkers and linking them to age may thus provide a basis for a platform providing integrative analysis of health status, assess data quality, and identify fake data. In addition, treating aging as a disease to train the deep neural networks (DNNs) to capture the most important biological properties of the age-related changes that transpire during aging using the DNs facilitates transfer learning on individual diseases using a much smaller number of samples. This technique may be used to reconstruct data sets with missing or incorrect features.

Aging is also a continuous process gradually leading to loss of function and the age-associated diseases. The DNNs may be trained on multi-modal health data items 122, 124, 324, 326, 328 ranging from photographs, videos, blood tests, “omics,” activity, and even smell and sweat during aging may capture biologically-relevant features about the group, individual, organ, tissue, or even a set of molecules. These DNNs may be used to extract features from health data items 122, 124, 324, 326, 328 most implicated in aging and specific diseases to be used as targets or to build association networks and causal graphs. These DNNs may also be re-trained on a much smaller number of data sets of specific diseases within the same health data item 122, 124, 324, 326, 328 type or using multiple types of health data items 122, 124, 324, 326, 328.

A high-level architecture using health data items 122, 124, 324, 326, 328 of various types may be able to perform such functions. For example, for each health data item 122, 124, 324, 326, 328 type, a DNN predictor of chronological age may be built using health data items 122, 124, 324, 326, 328 for reasonably healthy individuals. Individual DNNs may help detect outliers and enhance data quality controls and may then be used to train a multi-modal one-shot learning DNN. This architecture allows not only for accurate age prediction, but also for feature importance analysis. Results of such analysis across all predictors may determine an importance of each individual biomarker and may inform its relative affect on a predicted equivalent age for a patient. Multiple potential biomarkers related to age (e.g., albumin, glucose, norepinephrine, white blood cell count) are measured routinely in the clinic in separate tests of differing degree of invasiveness. It is therefore important to know which biomarkers are most predictive and thus most worth a patient undergoing to generate health data items 122, 124, 324, 326, 328 for upload and processing in generating an equivalent age for the patient.

### CONCLUSION

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A system comprising:
a health data storage configured to store health data items associated with a plurality of users; and
a user access portal configured to transmit an uploaded health data item to the health data storage and add a data upload transaction to a blockchain identifying a storage location of the uploaded health data item within the health data storage.

2. The system of claim 1, wherein the blockchain is implemented by a plurality of nodes that verify transactions using a consensus algorithm before storing the transactions on the blockchain.

3. The system of claim 1, further comprising a validator configured to perform an upload validation operation on the uploaded health data item and add an upload validation transaction to the blockchain reflecting a result of the upload validation operation performed on the uploaded health data item.

4. The system of claim 3, wherein the validator is configured to analyze the uploaded health data item with the upload validation operation to verify a data quality of the uploaded health data item before storing the uploaded health data item in the health data storage

5. The system of claim 1, further comprising a customer portal configured to grant and remove a customer access to a purchased health data item from the stored health data items

6. The system of claim 5, wherein the customer portal is further configured to generate a permission assignment transaction identifying (i) the customer, (ii) the purchased health data item, and (iii) an access permission level granted to the customer for the purchased health data item.

7. The system of claim 1, further comprising a key management system configured to (i) split a private key associated with the uploaded health data item into a plurality of key parts and (ii) store the plurality of key parts on a plurality of key holders, wherein the private key is used to encrypt the uploaded health data item to create an encrypted health data item before storing the encrypted health data item within the health data storage.

8. The system of claim 7, wherein the key management system is further configured to (i) combine the key parts to reconstruct the private key and (ii) decrypt the encrypted health data item upon receiving a download request for the uploaded health data item.

9. The system of claim 1, wherein the health data storage is also configured to store access permission levels granted to one or more users for one or more of the health data items.

10. The system of claim 9, wherein the access permission levels include one or more permissions from the group consisting of: (i) read access to the stored health data items, and (ii) read access to data resulting from calculations performed on the stored health data items.

11. A method comprising:
receiving a health data item;
storing the health data item on a health data storage;
generating a data upload transaction indicating the health data item and a user associated with the health data item; and
storing the data upload transaction on a blockchain.

12. The method of claim 11, further comprising:
verifying the data upload transaction with a plurality of nodes, wherein the nodes are configured to implement the blockchain.

13. The method of claim 11, further comprising:
validating a data quality of the health data item with an upload validation operation;
generating an upload validation transaction indicating the health data item and an upload validation result of the upload validation operation; and
storing the upload validation transaction on the blockchain.

14. The method of claim 11, further comprising:
receiving a data access request from a customer requesting access to the health data item;
generating a permission assignment transaction indicating the customer, the health data item, and a permission level granted to the customer; and
storing the permission assignment transaction on the blockchain.

15. The method of claim 14, further comprising:
comparing the data access request to a privacy setting of the user; and
determining that the data access request complies with the privacy setting of the user.

16. The method of claim 14, wherein the data access request includes a request for a plurality of health data items meeting one or more request criteria.

17. The method of claim 14, further comprising:
retrieving the health data item from the health data storage;
validating the health data item with a download validation operation;
generating a download validation transaction indicating the health data item, the customer, and a download validation result; and
storing the download validation transaction on the blockchain.

18. The method of claim 17, wherein the health data item is encrypted on the health data storage, and wherein the method further comprises:
decrypting the health data item;
encrypting the health data item with an encryption key associated with the customer to create an encrypted health data item; and
providing the encrypted health data item to the customer.

19. The method of claim 11, further comprising:
receiving a private key associated with the uploaded health data item;
splitting the private key into a plurality of key parts; and
storing the plurality of key parts in a plurality of key holders.

20. The method of claim 19, further comprising:
retrieving the plurality of key parts from the key holders; and
reconstructing the private key from the plurality of key parts.
